(19) United States
(12) Patent Application Publication
(10) Pub. No.: US 2006/0051789 A1
(43) Pub. Date: Mar. 9, 2006

(54) Methods of preparation of gene-specific oligonucleotide libraries and uses thereof

(75) Inventors: Sergei A. Kazakov, Los Gatos, CA (US); Alexander V. Vlassov, Santa Cruz, CA (US); Anne Dallas, Santa Cruz, CA (US); Attila A. Seyhan, San Jose, CA (US); Levente A. Egry, Santa Cruz, CA (US); Heini Ilves, Santa Cruz, CA (US); Roger L. Kaspar, Santa Cruz, CA (US); Brian H. Johnston, Scotts Valley, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Somagenics, Inc.

(21) Appl. No.: 11/173,100

(22) Filed: Jul. 1, 2005

Related U.S. Application Data
(60) Provisional application No. 60/585,035, filed on Jul. 1, 2004.

(51) Int. Cl. C40B 40/08; C12P 19/34

(52) U.S. Cl. 435/6; 435/91.2

(57) ABSTRACT

Methods of preparing gene-specific oligonucleotide libraries are disclosed. In one embodiment a double-stranded RNA corresponding to both sense and antisense strands of mRNA is digested by ribonuclease to produce short RNA fragments. In subsequent ligation steps, flanking oligoribonucleotides of defined sequences may be attached to the 3- and 5-ends of each fragment by RNA ligase (such as T4 RNA ligase). The products of ligation can be reverse transcribed and PCR amplified (RT-PCR) using the oligonucleotides attached to the gene-derived sequences as primer-binding sites. Various methods for incorporating libraries into expression vectors allowing expression of either siRNAs or shRNAs are also disclosed.

### FIELD OF THE INVENTION

The invention provides methods and reagents for producing gene-specific (directed) oligonucleotide libraries comprising sequences of defined length corresponding to portions of a polynucleotide target of interest, and their uses in wide range of nucleic acid applications, as gene inhibitors and analytical/diagnostics probes.

### BACKGROUND OF THE INVENTION

Important requirements for gene inhibitors and diagnostic methods based on hucleic acids are sequence specificity and high efficacy. Such applications include si/shRNA (small interfering/small hairpin RNA) (Rossi et al. (2002) Nucleic Acids Res. 30:1757-1766; Shi (2003) TRENDS Genetics 19: 9-12; Bohula et al. (2003) J. Biol. Chem. 278: 15991-15997), ribozyme (Scarabino & Tocchini-Valentini (1996) FEBS Lett. 383:185-190; Amarzguioui et al. (2000) Nucleic Acids Res. 28:4113-4124), and antisense (Bruice & Lima (1997) Biochemistry 36:5004-5019; Sohail & Southern (2000) Adv. Drug Deliv. Rev. 44:23-34) approaches to gene inhibition, as well as microarrays (Southern et al. (1999) Nat. Genet. 21:5-9), competitive RT-PCR (Ishibashi (1997) J. Biochem. Biophys. Methods 35:203-207), blots and in situ hybridization.

The specificity and efficacy of probe hybridization depends on parameters such as target accessibility, hybridization rate, and the stability of the formed duplex (Sczakiel and Far (2002) Curr. Opin. Mol. Ther. 4:149-153). Because of the complexity of these interactions, the rational design methods, both experimental and theoretical, that have been developed for predicting optimal probe sequences and target site accessibility have had only limited success (Sczakiel & Far (2002) Curr. Opin. Mol. Ther. 4:149-153; Sohail & Southern (2000) Adv. Drug Deliv. Rev. 44: 23-34). Also, the common notion that sequences that are less involved in internal hydrogen bonding interactions represent more favorable target sequences is an oversimplification (Sczakiel & Far (2002) Curr. Opin. Mol. Ther. 4:149-153; Fakler et al. (1994) J. Biol. Chem. 269:16187-16194; Laptev et al. (1994) Biochemistry 33:11033-11039). Target RNAs are often folded differently in the cell than in vitro (Lindell et al. (2002) RNA 8:534-541), and may be complexed with proteins that further reduce target site accessibility (Lieber & Strauss (1995) Mol. Cell Biol. 15:540-551). Conversely, some cellular factors may promote probe hybridization with target sites that are not accessible in vitro (Laptev et al. (1994) Biochemistry 33:11033-11039; Bertrand & Rossi (1994) EMBO J. 13:2904-2912).

As a consequence of this complexity, optimal sequences of nucleic acid hybridization probes as well as antisense and ribozyme gene-inhibitors (drugs) cannot reliably be selected based on sequence data analysis or using experimentally-determined in vitro target accessibility. To address this problem, several in vitro and in vivo methods for selecting optimal target sequences from sequence libraries have been developed, using 5-30 nucleotide long variable sequences (Lieber & Strauss (1995) Mol. Cell. Biol. 15:540-551; Allawi et al. (2001) RNA 7:314-327; Lloyd et al. (2001) Nucleic Acids Res. 29:3664-3673; Ho et al. (1998) Nat. Biotechnol. 16:59-63; Birikh et al. (1997) RNA 3:429-437; Lima et al. (1997) J. Biol. Chem. 272:626-638; Wrzesinski et al. (2000) Nucleic Acids Res. 28:1785-1793; Scherr et al. (2001) Mol. Ther. 4:454-460; Milner et al. (1997) Nat. Biotechnol. 15: 37-541; Patzel & Sczakiel (2000) Nucleic Acids Res. 28: 2462-2466; Yu et al. (1998) J. Biol. Chem. 273:23524-23533; WO 00/43538; WO 02/24950). An additional advantage of such libraries is that they can be used in a “reverse” genomics approach, which can identify genes responsible for a specific phenotype without prior knowledge of any sequence information (Li et al. (2000) Nucleic Acids Res. 28:2605-2612; Kawasaki & Taira (2002) Nucleic Acids Res. 30:3609-3614) Akashi et al. (2005) Nature Rev. 6:413-22. In case of small interfering RNAs (including siRNA, shRNA and miRNA) the situation is even more complicated.

In the case of siRNAs and shRNAs, the situation is even more complicated. Not all siRNA and shRNA sequences are equally potent or specific. Although it has long been thought that siRNAs shorter than about 30 bp avoided induction of interferon and PKR, recent reports indicate that in fact siRNAs longer than about 19 bp (Fish & Kruithof (2004) BMC Mol. Biol. 5:9) or having a 5′-triphosphate group (Kim et al. (2004) Nat. Biotechnol. 22: 321-325) can trigger an interferon response. In addition, siRNAs can produce off-target effects, whereby unintended mRNAs are silenced due to having partial homology to the siRNA. Off-target effects may be less problematic with highly potent siRNAs because they can be used at lower concentrations, where discrimination between matched and mismatched targets is greater. Identifying highly potent siRNAs is also crucial to efforts to develop siRNA therapeutics. High potency has been associated with specific sequence features as well as the internal stability profile of the siRNA and the accessibility of the mRNA target site (Elbashir et al. (2001) Nature 411: 494-498; 2001; Lee et al. (2002) Nat. Biotechnol. 20: 500-505; Paul et al. (2002) Nat. Biotechnol. 20: 505-508; Paul et al. (2002) Nat. Biotechnol. 20: 505-508; Hohjoh (2002) FEBS Lett. 521: 195-199; Holen et al. (2002) Nucleic Acids Res. 30: 1757-1766 Khvorova et al. (2003) Cell 115: 209-216; Kretschmer-Kazemi et al. (2003) Nucleic Acids Res. 31: 4417-4424; Reynolds et al. (2004) Nat. Biotechnol. 22: 326-330; Ui-Tei et al. (2004) Nucleic Acids Res. 32: 936-948). These correlations have been incorporated into algorithms that are commonly used to predict functional siRNAs. Despite their success at finding good siRNAs, many effective siRNA sequences are not predicted by current algorithms. Ideally, all possible target-specific siRNA sequences of appropriate lengths would be tested in cells to assure finding the best inhibitors for a given mRNA (Singer et al. (2004) Proc. Natl. Acad. Sci. USA. 101: 5313-5314). However, such a “brute force” approach is expensive and time-consuming. An attractive alternative is to screen cell-based libraries of sequences for the most potent siRNAs, without any bias for or against sequence features except for their presence within the target.

In principle, screening for gene inhibitors may be performed by using completely random (degenerate) libraries. However, this approach has several major problems. The high complexity of random libraries (e.g., 4²⁰ or ˜10¹² molecules for 20-nt antisense sequences represented only about once in the human genome) (Saha et al.) may make this approach time-consuming and expensive for cell-based assays (Kruger et al., 2000; Kawasaki & Taira, 2002; Miyagashi & Taira, 2002; Tran et al. 2003). Also, experiments have shown that degenerate libraries are highly toxic to cells: antisense ribozymes with degenerate substrate recognition sites can efficiently block the functioning of both mRNAs of interest (host or foreign) and unintended cellular RNAs (Pierce & Ruffner, 1998; Kruger et al., 2000). Several groups have made gene-specific siRNA pools by digestion of long RNA duplexes with E. coli RNase III (Calegari et al. (2002) Proc. Natl. Acad. Sci. USA 99: 14236-14240; Yang et al. (2002) Proc. Natl. Acad. Sci. USA 99: 9942-9947; Yang et al. (2004) Methods Mol. Biol. 252: 471-482; Kittler et al. (2004) Nature 432: 1036-1040) or recombinant human Dicer (Kawasaki et al. (2003) Nucleic Acids Res. 31: 981-987). Such siRNA pools are able to efficiently silence target mRNAs, and can be directly used in cell-based loss-of-function studies. However, no selection of the most potent siRNA species is possible unless RNAs are converted into DNA sequences and incorporated into appropriate expression vectors (as described in the present invention). Such expression vectors may contain opposing (convergent) promoters, allowing transcription of both RNA strands, which can then anneal to form functional siRNA molecules. Similar vectors to express siRNA libraries comprising both defined and randomized sequences have been recently described (Tran et al. (2003) BMC Biotechnol. 3: 1-9; Zheng et al. (2004) Proc. Natl. Acad. Sci. USA. 101: 135-140; Seyhan et al. (2005) RNA 11: 837-846)

A number of previous studies have suggested that for a given target site, shRNAs expressed as single molecules from vectors with pol IlIl promoters are generally more effective than siRNAs expressed as separate strands from opposing promoters. Any effective siRNA sequences identified by screening of gene-specific siRNA libraries can be subsequently converted to the shRNA format and tested for improvements in gene silencing. However, in certain cases pol III-expressed siRNA libraries may have an advantage over shRNA libraries. Since short siRNAs may bypass the Dicer processing pathway (Lee et al. (2002) Nat. Biotechnol. 20: 500-505; Paul et al. (2002) Nat. Biotechnol. 20: 505-508; Miyagishi & Taira (2002) Nat. Biotechnol. 20: 497-500), siRNAs could potentially be used in differentiated cells containing little or no Dicer (Brummelkamp et al. (2002) Science 296: 550-553; Sui et al. (2002) Proc. Natl. Acad. Sci. USA 99: 5515-5520; Parrish et al. (2000) Mol. Cell. 6: 1077-1087; Zheng et al. (2004) Proc. Natl. Acad. Sci. USA. 101: 135-140). Besides, shRNAs can be difficult to amplify and transcribe, and are unstable during cloning in E. coli, which can lead to a reduction in library coverage and potential loss of the best target sites.

To take full advantage of the expressed siRNA libraries, an appropriate screen for the most potent siRNA species should be devised. The screening can be done by cloning all species and testing them individually in cell culture, a very laborious process (Zheng et al. (2004) Proc. Natl. Acad. Sci. USA. 101: 135-140; Aza-Blanc et al. (2003) Mol. Cell. 12: 627-637) or by a screen for the phenotype conferred by inhibition of the target. For fluorescent-tagged targets such as GFP fusions, a fluorescence-activated cell sorter can be used. For targets whose silencing confers a growth or survival advantage, such as a virus or a pro-apoptotic gene, the desired species will outgrow the others. For other targets, fusion with a “suicide gene” such as the thymidine kinase of Herpes simplex virus (HSV-TK) can also allow selection for cells in which the target is silenced (Shirane et al. (2004) Nat. Genet. 36: 190-196).

Directed (gene-specific) libraries comprised of all 15-25-nt long sequences represented within the target gene(s) of interest offer a superior alternative to screening completely random libraries. The use of directed libraries prepared in vitro significantly simplifies the screening process since comparatively small libraries need to be assayed. For example, a 20-nt directed library targeting a 2000-nt long mRNA consists of only 1981 different molecules. Moreover, unintended knockdown of non-targeted genes is reduced, allowing more efficient cell-based assays with the directed libraries cloned into appropriate vectors. Currently, there are several reported methods of preparation of directed libraries that can be cloned, amplified and inserted into appropriate antisense, ribozyme, or siRNA expression cassettes (Pierce & Ruffner, 1998; Ruffner et al., 1999; Paquin et al., 2000; Sohail & Southern, 2000; Kazakov et al., Vlassov et al. 2004).

One method that has been used for preparation of a directed sequence library is a multi-stage process for making a directed antisense library against a target transcript specifically for hammerhead ribozyme constructs (Pierce and Ruffner (1998) Nucleic Acids Res. 26:5093-101; WO 99/50457). This method involves multiple enzymatic manipulations to produce a directed library of antisense sequences with a uniform length (10 or 14 nt, determined by the type IIS restriction endonuclease used in the procedure). In addition to the technical complexity of the procedure, this method has the additional disadvantage that the terminal ˜500 nucleotides at each end of the target sequences are missing, and the size of the antisense sequences is restricted to a 14-nt or less (which is less that than required for siRNAs).

Another method for producing a directed library, described in WO 00/43538 and Bruckner et al. (2002) Biotechniques 33: 874-882, includes hybridization of an immobilized DNA target with a randomized sequence of uniform length (20 nucleotides), flanked on each end by a defined primer sequence masked by complementary blocking oligonucleotides. This method suffers from several serious drawbacks: the complexity of the initial random library (4²⁰ or 10¹²) is higher than any target gene complexity (and even the entire human genome). The screening of such libraries is very time- and labor-intensive, and it requires immobilization of the target polynucleotides. The method is restricted to the use of long, immobilized DNA targets, which hybridize to oligonucleotide probes less efficiently than shorter, non-immobilized oligonucleotide fragments in solution (see, e.g., Armour et al. (2000) Nucleic Acids Res. 28: 605-09; Southern et al. (1999) Nature Genet. Suppl. 21:5-9). Hybridization with an immobilized target requires large volumes for hybridization solutions. Solid-phase hybridization methods produce high background due to nonspecific surface effects. Extra steps are required to separate bound from unbound probes and to elute bound probe from the target prior to amplification of the bound sequences. In addition, hybridization patterns obtained with a completely random 20-nucleotide library are expected to be far less intense than those obtained with shorter libraries, due to formation of complementary complexes among members of the library (see, e.g., Ho et al. (1996) Nucleic Acids Res. 24:1901-07). Even when a high initial concentration of the 20-nucleotide random library is used, the concentration of individual sequences in the random pool is not high enough to provide efficient hybridization with a DNA target (see, e.g., Wertmur (1991) Critical Rev. Biochem. Mol. Biol. 26:227-59). Finally, the method has low specificity; WO 00/43538 suggests that the majority of the 20-mer sequences captured on an immobilized DNA target from the random oligonucleotide pool at 52° C. will contain 4-8 mismatches.

Yet another method that has been used is described in Boiziau et al. (1999) J. Biol. Chem. 274: 12730-12737, using a “template-assisted combinatorial strategy”. Boiziau et al. selected DNA aptamers targeting an accessible binding site in an RNA hairpin, using both completely random libraries and libraries “enriched” in target-specific sequences. The “enriched sequences” were produced by ligation of “half-candidates” in the presence of an RNA hairpin using RNA ligase. The half-candidates were designed as hemi-random probes containing defined primer and comparatively long 15-nt terminal random sequences, and were used without masking oligonucleotides in the ligation reaction. Both ligation methods showed low efficiency and target-specificity, which is a consequence of the preference of RNA ligase to ligate sequence motifs that are not aligned in complementary complexes (Harada and Orgel (1993) Proc. Natl. Acad. Sci. USA 90: 1576-1579. Also, due to the lack of masking oligonucleotides, most ligation products were unrelated to the RNA target. Consequently, the authors found no benefit to using libraries prepared from hemi-random probes versus using probes with completely random 30-mer libraries without a ligation step.

Recently, Shirane et al. (Shirane et al. (2004) Nat. Genet. 36: 190-196) developed another method of preparation of a directed library of 19-21 bp DNA fragments that allows expression of shRNA from the library. This method includes quasi-random fragmentation of a double-stranded DNA corresponding to the gene of interest by DNase I (Matveeva et al. 1997). The ends of these fragments were blunted by DNA polymerase and ligated by DNA ligase to a hairpin-shaped adaptor containing the recognition sequence of Mme I restriction endonuclease. Subsequent cleavage by Mme I produced DNA fragments of uniform length of 19-21 bp. This preparation scheme is rather complex, and the obtained library is restricted to species ˜20 nt in length.

Alternatively, the same enzyme Mmel was used to adjust the length of double-stranded DNA fragments of a gene of interest produced by action of mixture of restriction endonucleases including HinpI, BsaHI, Acil, HpaII, HpyCHIV and Taqαl (Sen et al. (2004) Nat. Genet. 36: 183-189). These restrictases are frequent cutters and leave identical CG-overhangs to facilitate cloning. In the next step of this scheme, the obtained DNA fragments were ligated to the loop sequence containing the Mmel restriction site, which was used to generate ˜20 bp long fragments of the directed library. Using a multi-step procedure, the resulting fragments were cloned into expression vectors to produce the shRNA library. The main drawback of this scheme is that the cocktail of restriction enzymes does not produce sufficiently random cuts, and as a result the obtained library contained only 34 unique target-specific sequences out of theoretically possible 981 for the 1000-nt long target. This too is a rather complex scheme and the obtained library is also restricted in length to ˜20 nt.

In view of the foregoing, there is a need for an improved procedure for generating a directed sequence library that is highly specific for the target sequence from which the library is generated, and that does not suffer from the limitations of the methods described above. Also, there is a high demand for improved cassettes to express directed libraries and subsequent selection schemes allowing to choose the best candidates, including antisense RNA, ribozymes, si/shRNA.

### SUMMARY OF THE INVENTION

Methods are provided for producing target-specific (directed) libraries that comprise substantially all sequences of a pre-determined length that are comprised within a target polynucleotide sequence, which polynucleotide may be a gene, plurality of genes, genome, etc. Such libraries are useful in the expression and selection of gene expression inhibitors and molecular tools, analytical assays and diagnostics specific for the target polynucleotide.

In one embodiment of the invention, a double-stranded RNA comprising complementary strands of a target polynucleotide is digested by ribonuclease to produce double stranded RNAs of a predetermined size. In some embodiments, the RNAse is a length-directed RNAse, e.g. Dicer, which may be utilized in combination with an enzyme providing 3′ phosphatase activity, e.g. ExoIII. The dsRNA fragments of pre-determined size are ligated to oligoribonucleotides of defined sequence at both the 3′- and 5′-ends. The products of ligation are reverse transcribed and amplified using the ligated oligonucleotides as primer-binding sites.

In another embodiment of the invention, a directed library is produced by ligation of hemi-random probes hybridized to adjacent sites on a polynucleotide target. After ligation of the probes with a DNA ligase (such as T4 DNA ligase), pairs of ligated probes are PCR amplified.

In yet another embodiment, a deoxyribonuclease (e.g. DNase I) is used to digest the target polynucleotide. Flanking oligonucleotides are ligated to the obtained fragments, allowing subsequent PCR amplification using the oligonucleotide sequences as primer-binding sites.

The amplified double-stranded DNA fragment encoding the directed libraries, obtained by any of the above described methods, can be inserted in an expression cassette, where such cassettes include PCR templates, vectors, etc. Various methods can be used for this purpose, including annealing to flanking oligonucleotides and extension with Klenow polymerase (in case of PCR cloning); enzymatic ligation using blunt ends or specific restriction sites; and the like. In the latter case, treatment of the amplified polynucleotides with restriction endonucleases (acting at sites encoded in primer-binding flanking constant regions) releases directed sequence inserts.

The directed libraries are useful in various screening methods. The expressed RNA may be selected for functional characteristics, including efficacy as antisense, ribozyme, siRNA, shRNA, miRNA; etc. can be expressed, according to suggested protocols. Selection schemes of interest include, without limitation, selection of RNA Lassos capable of fast and efficient hybridization with target RNA; selection of potent inhibitors from siRNA libraries in vivo; selection of optimal viral target sites in virus-infected mammalian cells; and the like.

These and other objects, advantages, and features of the invention will become apparent to those persons skilled in the art upon reading the details of the methods of producing libraries and uses thereof as more fully described below.

### DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIGS. 1A-1B schematically depict preparation of a directed library from an siRNA pool obtained by Dicer (or RNase III)-digestion of target-encoding dsRNA. (A) The general scheme. The double-stranded RNA target is digested by Dicer (or RNase III) to produce 20-22 bp siRNAs. In two subsequent ligation steps, single-stranded RNA adapters are attached to the 3′- and 5′-ends of each fragment by T4 RNA ligase. The products of ligation are reverse transcribed and PCR amplified using the oligonucleotides attached to the gene-derived sequences as primer-binding sites. The resulting PCR products are cut with appropriate restriction enzymes and cloned into the siRNA expression vector pU6/H1-coh (see FIG. 15). (B) Sequencing results for the randomly selected clones from the TNF-specific library.

FIGS. 2A-2B schematically depict production of a directed sequence library by ligation of hemi-random probes hybridized to a polynucleotide target. (A) Experimental scheme. After joining of the probes hybridized to adjacent positions on a polynucleotide target with a ligase, pairs of ligated probes are PCR amplified. Further treatment of the amplified polynucleotides with restriction endonucleases releases amplified directed sequence (both sense and antisense) inserts, yielding a directed sequence library of sequences corresponding to the original target. (B) Sequencing results for randomly selected samples of a prepared TNF-specific directed library. Target-matching sequences are highlighted. Clones #1-12: effect of competing random tetramer+5 mM spermidine on the quality of the directed library (in terms of the number of mismatches); clones #13-20: effect of 5 mM spermidine.

FIGS. 3A-3B schematically depict preparation of a directed library from a dsDNA target fragmented by DNase I. (A) The general scheme. The double-stranded DNA target is digested by DNase I in the presence of Mn²⁺ ions, and the fraction containing 20-30 bp fragments is gel-purified. Next, double-stranded DNA adapters are attached to 3′- and 5′-ends by T4 DNA ligase, and the resulting fragments are amplified by PCR. Further, fragments are cut with appropriate restriction enzymes and cloned into pU6/H1-coh (see FIG. 15). (B) Sequencing results for the randomly selected clones from the DsRed-specific library.

FIGS. 4A-4C schematically depict selection of RNA Lasso species that bind to and circularize around target RNA. (A) Sequence and secondary structure of unprocessed Lasso containing directed library. The position of the primer that is used to selectively extend by RT-RCA the circularized (but not linear) Lassos is indicated (primer 1). (B) Self-processed circular Lassos bound to its complementary site in TNFα mRNA. The primers that are used to both amplify the RT-RCA product and to convert it into a T7 polymerase transcription template are indicated. (C) Selection scheme for Lasso species that bind to and circularize around target RNA.

FIG. 5. Sequencing results for randomly selected samples of antisense sequences derived from a TNF-directed library which were incorporated into an RNA Lasso and subjected to 3 rounds of in vitro selection for fast-hybridizing and self-circularizing Lassos.

FIG. 6. Analysis of selected Lasso transcripts and their binding to TNF-1000 target RNA. Either Lasso alone (lanes 1) or Lasso and target RNA (lanes 2-3) were incubated for 15 min at 37° C. in SB buffer (10 mM MgCl₂, 20% formamide, 50 mM Tris-HCl, pH 7.5). Reactions were quenched with loading buffer containing 90% formamide and 10 mM EDTA. For lanes 3, prior to loading, samples were subjected to heat treatment at 95° C. for 2 min followed by placement on ice. Lasso numbers correspond to those listed in FIG. 5. Products were analyzed by denaturing 5% PAGE (8M Urea). C, circular Lasso; HP, hemiprocessed Lasso; L, linear.

FIG. 7. Sequences and secondary structures of the selected RNA Lassos TNF13 (top) and TNF4 (bottom).

FIG. 8. Time courses of binding of the selected Lassos with target TNF-1000 RNA. ³²P-labeled Lassos were incubated either alone or with non-radioactive target RNA at 37° C. for the time periods indicated. Complex formation was carried out in 50 mM Tris-HCl (pH 7.5), 10 mM MgCl₂, 20% formamide. Reactions were quenched with formamide loading buffer containing 10 mM EDTA. Products were analyzed by 5% denaturing PAGE (8M Urea).

FIG. 9. Sequencing results for randomly selected samples of antisense sequences derived from a DsRed-directed library, which was incorporated into an RNA Lasso and subjected to 3 rounds of in vitro selection for fast-hybridizing and self-circularizing Lassos.

FIGS. 10A-10B schematically depict the design of an RNA expression cassette for preparation of gene-specific (directed) or randomized shRNA libraries. A, Scheme for incorporation of appropriately sized single-stranded DNA (ssDNA) fragments, comprised of either randomized sequences or sequences of the gene(s) of interest, into an shRNA expression cassette template. B, Scheme for using the template from A for preparing an shRNA expression cassette encoding a single promoter for RNA polymerase and directed or randomized shRNA libraries. For more details, see Example 6.

FIG. 11 schematically depicts insertion and direct TA-cloning off a gene-specific siRNA library, obtained by Dicer/RNase III digestion of target-encoding dsRNA, into an expression vector between two opposing pol III promoters.

FIG. 12 schematically depicts conversion of directed libraries, obtained by one of the methods shown in FIGS. 1-3 (or by their combination), into hairpin and dumbbeII DNAs, followed by their PCR amplification and cloning under pol III (or pol II) RNA polymerase promoter for expression of shRNA directed libraries targeting gene(s) of interest. For more detail description, see Example 8 below.

FIG. 13A-13B schematically depicts conversion of a restriction fragment, encoding a directed library, into hairpin DNA and its PCR-assisted fusion with pol III promoter (U6 or H1), followed by cloning into a vector to express an shRNA library. The dsDNA fragments are cut with Hind III and Bgl II and ligated to two linkers, one in the form of a hairpin (Cap) and the other a partial duplex DNA containing a 3′-tail that is complementary to the 3′-end of the h-U6 promoter. This product is then used as a reverse primer alongside a primer specific to the 5′-end of the U6 promoter, resulting in a U6 transcription cassette. The PCR product is ligated into pCRII plasmid or viral vectors. Vectors are digested with Bgl II to remove the extraneous sequences flanking the loop and religated, forming the final product, expression-ready shRNA vectors. The transcribed shRNA is shown at the bottom.

FIG. 14A-B schematically depicts conversion of the fusion product between a pol III (U6 or H1) promoter and a restriction fragment, encoding a directed library, into a dumbbell-shaped DNA followed by its RCA amplification and cloning into vector to express shRNA or siRNA library.

FIGS. 15A-15B. Scheme for expression of siRNA libraries from opposing pol III promoters. (A) U6/H1 expression cassette used for cloning of cohesive-ended fragments (pU6/H1-coh; modified from Zheng et al. 2004). (B) The U6/H1 expression cassette allowing blunt-end cloning of siRNA library inserts (pU6/H1-blunt).

FIGS. 16A-16B. Silencing ability of species randomly selected from the TNF-specific siRNA library produced by Dicer method. (A) Randomly chosen clones were cotransfected with a TNF expression vector and pSEAP into 293FT cells with Lipofectamine 2000 (Invitrogen). TNF was assayed by ELISA and SEAP by a colorimetric assay 48 h post-transfection. The inhibition by each siRNA is shown, normalized to the SEAP control target. Rationally designed control shRNAs targeting TNF (shRNA-TNF-229) and DsRed (shRNA-DsRed-2) were expressed from pU6. Rationally designed control siRNAs targeting TNF (siRNA-TNF-229) and DsRed (siRNA-DsRed-2) were expressed from pU6/H1. (B) Representative sequences of the assayed clones.

FIGS. 17A-17B. Silencing ability of species randomly selected from the DsRed-specific siRNA library produced by the DNase I method. (A) Randomly chosen clones were cotransfected with DsRed expression vector into 293FT cells with Lipofectamine 2000 (Invitrogen). DsRed protein levels were quantified by flow cytometry 48 h after transfection. Cells were also imaged by fluorescence microscopy. The amount of inhibition of each siRNA was normalized to the pU6/H1 empty vector. Rationally designed control siRNAs targeting DsRed (siRNA-DsRed-2) TNF, (siRNA-TNF-229) and eGFP (siRNA-eGFP) were expressed from pU6/H1. Rationally designed control shRNA targeting DsRed (shRNA-DsRed-2) was expressed from pU6. (B) Representative sequences of the assayed clones.

FIG. 18. Scheme for selection of optimal viral target sites in virus-infected mammalian cells. Transduction of target cells with the RNA inhibitor vector library using lentiviral vectors results in stable cell lines expressing RNA inhibitor transcripts. These cells are challenged with infectious virus and surviving cells are collected and propagated. Putative antiviral sequences are rescued from the surviving cells and further analyzed to identify potential target genes using antisense sequence information.

FIG. 19. Scheme for selecting potent inhibitors from siRNA libraries in vivo. Stable transfection of target cells with the TK/DsRed/DV construct results in cells susceptible to complete killing with ganciclovir. Prior to ganciclovir treatment, the cells are transfected with the siRNA library. Following challenge with ganciclovir, surviving cells are collected and propagated. Putative antiviral siRNA species rescued from the surviving cells are purified and analyzed to identify the most potent siRNA species.

### DETAILED DESCRIPTION OF THE INVENTION

Before the present methods, libraries, and uses thereof are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a sequence” includes a plurality of such sequences and reference to “the ligation” includes reference to one or more ligations and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### General Techniques

The practice of the present invention will employ, unless otherwise indicated, conventional techniques of molecular biology (including recombinant techniques), microbiology, cell biology, and biochemistry, which are within the skill of the art. Such techniques are explained fully in the literature, such as: “Molecular Cloning: A Laboratory Manual,” vol. 1-3, third edition (Sambrook et al., 2001); “Oligonucleotide Synthesis” (M. J. Gait, ed., 1984); “Methods in Enzymology” (Academic Press, Inc.); “Current Protocols in Molecular Biology” (F. M. Ausubel et al., eds., 1987); “PCR Cloning Protocols,” (Yuan and Janes, eds., 2002, Humana Press).

### Production of Directed Sequence Libraries Based on Length Specific RNAse Digestion of a dsRNA Target Polynucleotide

The invention provides a method that produces essentially perfect directed libraries, comprising substantially all sequences of a pre-determined length that are comprised within a target polynucleotide sequence. By producing a substantially complete library of defined length fragments, the target polynucleotide is efficiently analyzed for fragments corresponding to optimal sequences for various purposes, such as RNA Lasso; siRNA; ribozymes; and the like. By “substantially all”, it is intended that the library comprises at least about 90% of the possible sequences, and may comprise at least about 95%, at least about 99%, or more.

Target polynucleotides of interest include RNA species, e.g. mRNA, groups of mRNAs, etc., and DNA species, e.g. genes, introns, exons, regulatory sequences, genomes of mitochondria, viruses, bacterial, eukaryotes, etc.

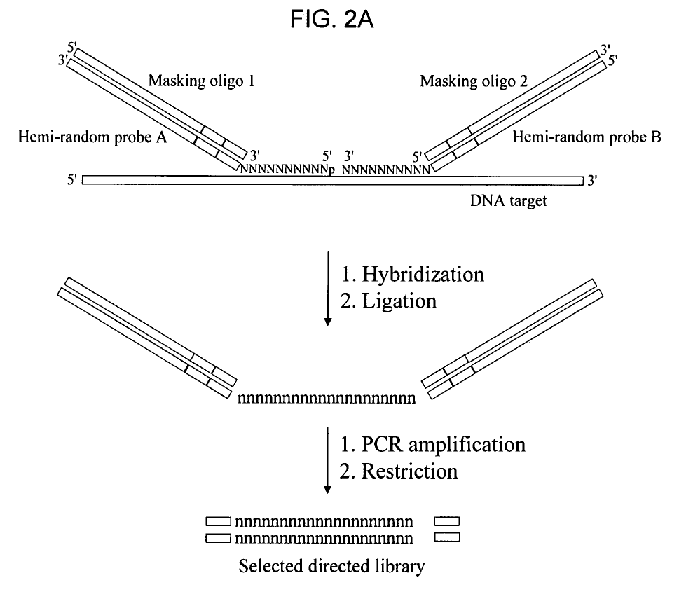

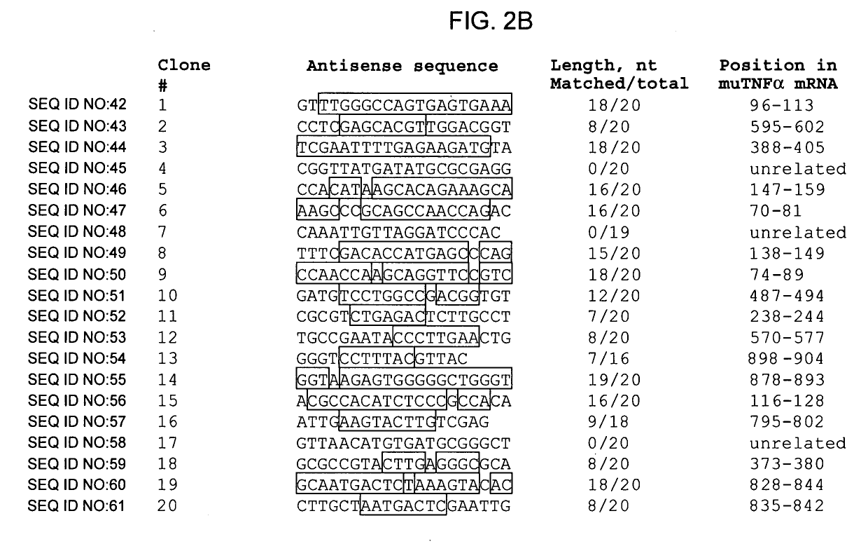

In some embodiments of the invention, enzymatic reactions are performed on dsRNA species as schematically shown in FIG. 2A. The target polynucleotide may be converted from a DNA strand or strands or an RNA strand into a dsRNA strand by any convenient method known in art. Transcription of RNA from a template is well known in the art. One of skill in the art will readily utilize opposite facing promoters in an expression cassette to produce complementary RNA strands. Any suitable promoter may be utilized, preferably one having high activity in an in vitro system, e.g. SP6, T7, T3, etc., where the two promoters may be the same or different, usually different. The RNA polymerase or polymerases will be selected to be appropriate for the promoters. Expression cassettes may be linear or circular, and may be present in a vector, in a PCR derived template, and the like. Separate reactions are optionally utilized for transcription of the two strands. The complementary RNA strands are annealed to form a dsRNA molecules (for example, see Kawasaki et al. (2003)).

The resulting dsRNA is nuclease digested. In some embodiments, the nuclease is a length-directed RNAse, where for the purposes of the present invention, a length-directed ribonuclease cleaves an RNA, usually a dsRNA, into fragments of defined length greater than about 10 nucleotides in length, usually in a processive manner. The length is usually at least about 10 nucleotides, more usually at least about 12 nucleotides, and may be at least about 20 nucleotides; and not more than about 40 nucleotides, more usually not more than about 30 nucleotides, and may be not more than about 25 nucleotides. In other embodiments, the nuclease is not length-directed and the resulting digestion product is size fractioned prior to use, e.g. by gel electrophoresis, etc. Preferred nucleases cleave in a non-site specific manner.

Length-directed nucleases of particular interest for this purpose are Dicer and RNAse III. Both recombinant human Dicer and Escherichia coli RNase III can be used in vitro to cleave long dsRNA. Dicer is an endoribonuclease that contains RNase III domains and is the enzyme responsible for cleavage of long dsRNAs to siRNA in the endogenous RNAi pathway. The siRNAs produced by Dicer are about 19-21 bp in length and contain 3′ dinucleotide overhangs with 5′-phosphate and 3′-hydroxyl termini (Myers et al. 2003; Kawasaki et al. 2003, supra). E. coli RNase III is involved in the maturation and degradation of diverse cellular, phage, and plasmid RNAs. Also applicable for digesting long dsRNA, its cleavage products range from ˜11-25 bp in length with termini identical to those produced by Dicer (Yang et al. 2002; Yang et al. 2004). Both ribonucleases are commercially available from multiple sources.

When provided short targets (<65 bp), Dicer appears to measure from an end in determining its cut sites (Zhang et al. (2002) EMBO J. 21: 5875-5885; Zhang et al. (2004) Cell 118: 57-68; Siolas et al. (2004) Nat. Biotech. 23:227-231), raising the question of whether sequential cut sites in longer RNAs are in register and might skip over some target sequences. The fact that digestion from either end can occur in most cases provides a second register of cutting which reduces the likelihood of skipping some sequences. Moreover, since each cut site is actually a distribution of several adjacent cleavages (see Zhang et al. (2004), supra), each successive cleavage makes the distribution wider and wider, so that essentially all sites are cleaved except those within about 60-100 bp of the ends. By starting with a dsRNA target flanked by extra 100 bp of nontarget sequences at either end, this concern can be eliminated, and the resulting addition of a few nontarget siRNAs to the library will have no effect on the effectiveness of library screening. In some embodiments of the invention, the target nucleic acid is flanked by at least about 60 nucleotides, and may be flanked by 100 nt. or more of nontarget sequence.

The fact that Dicer cleaves longer dsRNAs more efficiently than shorter ones (Bernstein et al. (2001) Nature 409: 363-366; Elbashir et al. 2001, supra; Ketting et al. (2001) Genes & Dev. 15: 2654-2659) suggests that this enzyme may have “endonuclease” activity, independent of ends and therefore not in any fixed register, that is not evident with short fragments where end effects may dominate. Alternatively, fragmentation of a DNA target by DNase I avoids end effects since that enzyme is a true endonuclease. Some sequence preferences can be seen with light digestion (Herrera and Chaires (1994) J. Mol. Biol. 236:405-411), so adjusting the level of digestion to provide fragments mostly shorter than 30 bp would further reduce the likelihood of missing any sequences in the final library.

The digestion product of the RNAse digestion comprises small dsRNA fragments, which may be of a defined size. The fragments are strand-separated, and may be purified by length, e.g. gel electrophoresis, capillary electrophoresis, HPLC, etc. The fragments are dephosphorylated, e.g. by alkaline phosphatase.

In ligation steps, flanking oligoribonucleotides of defined sequences are attached to the 3′-and 5′-ends of each fragment by T4 RNA ligase. Similar ligation-amplification methods have been previously used for cloning of small RNA fragments extracted from cells (Elbashir et al. 2001; Lau et al. 2001; Pfeffer et al. 2003). The flanking oligonucleotides provide primer-binding sites for the PCR amplification that will take place on the last stage of the protocol. These oligonucleotides also may provide restriction sites.

The reaction may be optimized to prevent circularization via intramolecular ligation of the oligonucleotides during the ligation reaction by the following steps. In a first ligation reaction, a first flanking oligoribonucleotide is used, in which the oligoribonucleotide, comprises a 5′-phosphate and 3′ “terminator nucleotide”. A terminator nucleotide refers to a nucleotide containing a chemical modification at the 3′ end that prevents normal polymerization or ligation of the nucleotide into a polymer. Such terminator nucleotides may retain the ability to form base pairs, and may be recognized by enzymes that act on polynucleotides.

Such terminator modifications are known in the art, and include, without limitation: 2′,3′ dideoxythymidine; 2′,3′ dideoxycytidine; 2′,3′ dideoxyuridine; 2′,3′ dideoxyguanosine; 2′,3′ dideoxyadenosine. Any of the bases may be modified by addition of an alkyl spacer at the 3′ end, which inactivates the 3′ OH towards enzymatic processing. One of skill in the art will recognize that such spacers may be variable in the length of the carbon chain, e.g. 1, 2, 3, 4, 5 carbons, etc. Inverted bases, such as inverted dT, when incorporated at the 3′-end of an oligo lead to a 3′-3′ linkage which inhibits degradation by 3′ exonucleases and extension by DNA polymerases and ligases. 3′-O-methyl-dNTPs are described by Metzker et al. (1994) Nucleic Acids Res. 22(20):4259-4267. A large number of other modified or capped nucleotides have been described in the art, and may be used in the methods of the invention.

Following ligation to the first flanking ribooligonucleotide, the ligation product may be purified by any convenient method, e.g. gel electrophoresis, dialysis, capillary electrophoresis, HPLV, etc. The purified ligation product is then phosphorylated and ligated to a second flanking oligoribonucleotide lacking a terminal phosphate. In this second ligation reaction, the circularization of the product is prevented due to the absence of 5′-phosphate.

The ligation product of the second reaction is reverse transcribed and PCR amplified (RT-PCR) using methods known in the art, using the first and second flanking oligonucleotides as primer-binding sites. The resulting PCR-amplified DNA fragments may be used for various purposes, e.g. inserting into vectors for library generation, expression, sequencing, etc.

The directed libraries produced by this method contain both sense and antisense gene-specific sequences. If it is desirable to obtain sequences that correspond only to the antisense strand, this double-stranded RNA library can be denatured, the sense sequences annealed with an excess of the gene-specific antisense cDNA, and the unhybridized single-stranded antisense RNA fragments separated by a gel-electrophoresis or affinity chromatography and purified.

Alternative Method #1 for Directed Library Preparation Based on Ligation of Hemi-Random Probes on a ssDNA Target

An alternative method to prepare a gene-specific (directed) library, based on the hybridization of hemi-random probes to a ssDNA target with subsequent enzymatic ligation of the probes that happen to hybridize to adjacent target sequences (see FIG. 2A; Kazakov et al., International Patent Application (PCT): WO 03/100100 A1; Kazakov et al., 2004). The hemi-random probes contain fixed sequences consisting of primer-binding sequences with encoded restriction enzyme recognition sites and a 10-nt randomized sequence located either at the 5′-(probe A) or 3′-end (probe B). Masking oligonucleotides complementary to the constant regions of the hemi-random probes are employed to reduce false-positive, target-independent self-ligation of probes. The inclusion of competing oligoribonucleotides and/or spermidine in the reaction buffer increases the average length of match between probe and target. The hemi-random probes are annealed with the DNA target, and T4 DNA ligase is added. The ligated product is exponentially amplified by PCR using primers complementary to the constant regions of the probes A and B. This method, which relies on the fidelity of both hybridization and enzymatic ligation, has clear advantages over approaches based only on competing hybridization (Paquin et al., 2000; Brukner et al., 2002; Liang et al., 2002) in terms of sequence-specificity and the number of mismatches to the target sequences. However, even with this improved method, at least several mismatches occurred in the majority of the identified sequences, and thus the method produces a library of sequences highly related to and substantially enriched in target sequences, rather than a pure directed library.

Alternative Method #2 for Directed Library Preparation Based on DNase Fragmentation of a dsDNA Target

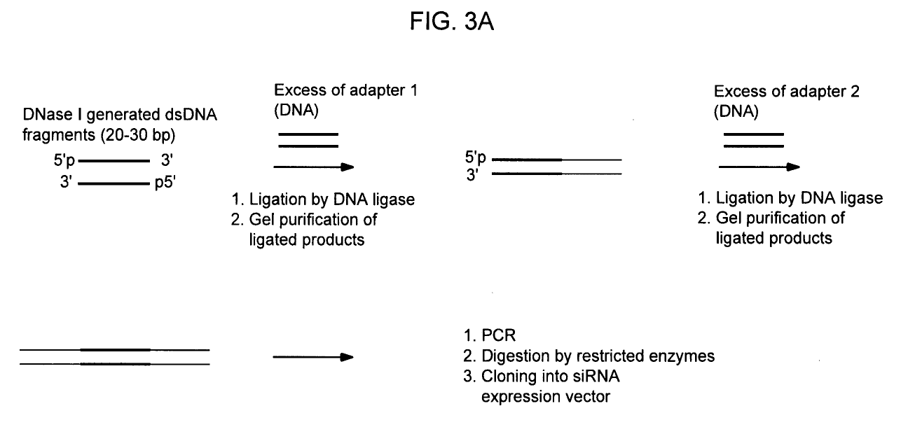

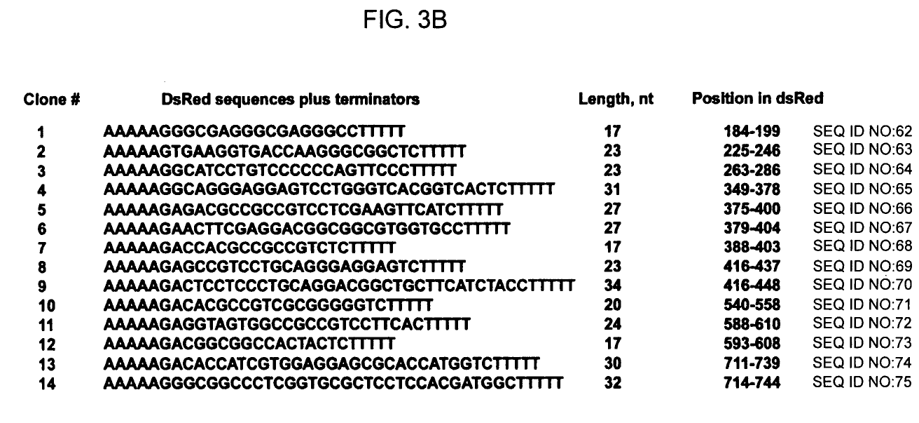

In this method, the directed libraries can be directly derived from gene-specific double-stranded DNA as shown in FIG. 3A. In the presence of Mn²⁺ or when very high concentrations of the enzyme are used in the absence of monovalent cations, DNase I breaks both strands of DNA simultaneously at approximately the same site (Melgar and Goldthwait, 1968 Campbell and Jackson, 1980; Holzmeyer et al. 1992). Under these conditions the enzyme displays little sequence specificity and cleaves all regions of the DNA (except the terminal nucleotides) at similar rates. DNase I generates fragments with a wide distribution of sizes; therefore, a careful gel purification or some other means of size separation must be used to isolate the ˜15-30 bp fraction of interest. Further, linkers are used to equip blunt-ended termini of DNA with restriction sites to aid in cloning into appropriate siRNA expression vectors between opposing pol II or pol III promoters. In addition, linker attachment allows PCR amplification as was discussed above. The linkers are subsequently attached by means of T4 DNA ligase as shown in FIG. 3A.

The fragmentation of DNA targets by DNase I and isolation of fragments of about 20 bp for preparation of shRNA libraries has been recently described by others (Sen et al (2004) Nat. Genet. 36: 183-189; Shirane et al. (2004) Nat. Genet. 36: 190-196) or suggested (Taira & Miyagishi (2004) U.S. patent application US2004/0002077 A1.) In the present invention, we use a wider range of DNase I fragment sizes for the expression of siRNA. We also suggest an additional purification and amplification of the PCR-amplified product obtained from the original DNase digest. This additional step provides a higher yield and allows easy purification of DNA fragments of the desired length.

The Dicer and DNase I methods of target fragmentation can be considered complementary, with each having certain advantages and disadvantages. The Dicer/RNase III-generated fragments are of course the same length as in vivo products of Dicer processing and can be directly incorporated into the RISC complex. The DNase-generated gene fragments may be more useful for the preparation of shRNA libraries, since the stem length of potent shRNAs can vary from 21 to 29 bp, depending on the sequence (Paddison et al. (2004) Nature 428: 427-431). Formation of long RNA duplexes from the transcribed antisense and sense strands may sometimes be a challenge for the Dicer/RNase III approach when dealing with highly structured RNAs such as viral internal ribosome entry sites (IRES) elements. On the other hand, the DNase I approach requires at least two gel fractionation steps, and may use three or more (the third after ligation of adapters and PCR).

To provide additional sequence and size diversity, libraries made by each method may be mixed prior to insertion in an expression vector.

### Uses for Directed Sequence Libraries

Directed sequence libraries and methods of the present invention may be used as starting materials for a multitude of applications, including development of diagnostic reagents, therapeutic reagents (e.g., polynucleotide therapeutics), genomics tools, affinity reagents, and the like.

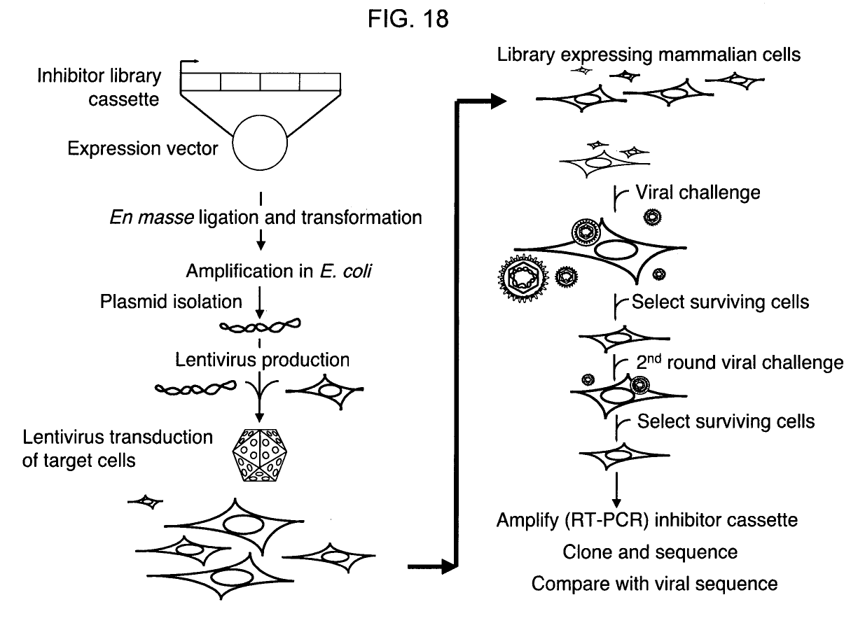

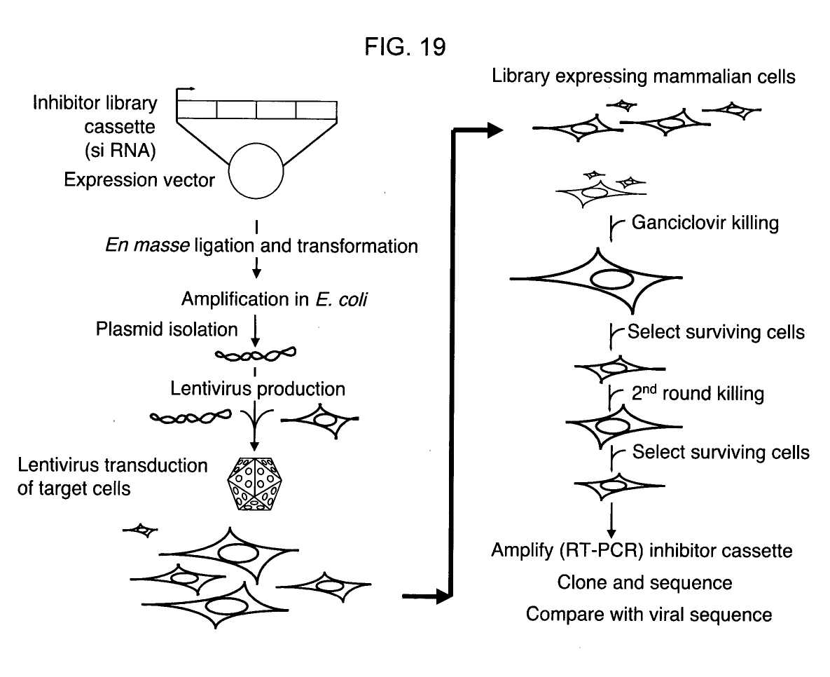

In one aspect, libraries of the invention are used (as alternative to fully random libraries) for development and optimization of sequences for antisense- and ribozyme-based polynucleotide genomics tools (e.g., gene knockdown, gene-target discovery and validation, etc.) and therapeutics by methods known in the art reviewed in references cited in the introduction. For example, a directed sequence library may be prepared from a gene sequence that provides a particular cellular function. Antisense sequences that block that function may be determined by screening the library for sequences that inhibit gene function. The screening can be performed in cells as described, for example, in paragraph [09], Examples 13 and 14, and FIGS. 18 and 19. Target accessibility, hybridization parameters, and inhibitory effects may also be assessed.

“Rationally-designed” nucleic acid therapeutics utilize various in silico algorithms known in the art to select a target site, and often are directed to a single site on the target RNA. Such therapeutics include antisense, ribozymes, deoxyribozymes, siRNA, shRNA and miRNA. In cases where the target mutates rapidly (e.g. HIV or influenza virus) the rationally-selected target sequences mutate over time, and the therapeutic becomes ineffective. The same is true for nucleic acid therapeutics directed at cancer targets, where mutations in a target sequence can lead to resistance to the nucleic acid therapeutic.

Nucleic acid therapeutics selected de novo from a pool of directed sequence libraries have advantages over those selected by in silico selection methods. Therapeutics selected from a directed sequence library of the invention complement multiple sites on a target simultaneously, allowing effective down-regulation of a rapidly mutating virus or cancer cell. Knowledge of the genetic sequence or molecular and structural biology of the virus or cancer cell are unnecessary, in contrast to rational drug design methods.

In another aspect, libraries of the invention are used for selection and optimization of sequences useful for RNA interference, such as siRNA (small interfering RNA) molecules capable of inhibiting known or unknown genes. “siRNA” refers to a double-stranded RNA molecule that inhibits expression of a complementary known or unknown gene(s) (see, e.g., Tuschl (2002) Nature Biotechnology 20:446-48).

In another embodiment, libraries of the invention are immobilized on a solid support to generate an array, which may be used to detect or quantify complementary polynucleotide sequences. The complete library may be used, or selection may be performed to optimize the array probes. Such arrays are useful in microarray-based diagnostics and gene expression analysis, including detection of the presence of bacterial and viral infectious agents, genetic traits and diseases, SNPs, etc. (see, e.g., Rampal, ed. (2001) DNA Arrays, Methods and Protocols (Humana Press).

As used herein, “microarray” refers to a surface with an array of putative binding (e.g., by hybridization) sites for a biochemical sample. Typically, a microarray refers to an assembly of distinct polynucleotides immobilized at defined positions on a substrate. Microarrays are formed on substrates fabricated with materials such as paper, glass, plastic (e.g., polypropylene, nylon), polyacrylamide, nitrocellulose, silicon, optical fiber, or any other suitable solid or semi-solid support, and configured in a planar (e.g., glass plates, silicon chips) or three-dimensional (e.g., pins, fibers, beads, particles, microtiter wells, capillaries) configuration. Polynucleotides may be attached to the substrate by a number of means, including (i) in situ synthesis (e.g., high-density polynucleotide arrays) using photolithographic techniques (see Fodor et al., Science (1991) 251:767-73; Pease et al., Proc. Natl. Acad. Sci. USA (1994) 91:5022-5026; Lockhart et al., Nature Biotechnology (1996) 14:1654; U.S. Pat. Nos. 5,578,832; 5,556,752; and 5,510,270); (ii) spotting/printing at medium to low density on glass, nylon, or nitrocellulose (see Schena et al., Science (1995) 270:467-70; DeRisi et al., Nature Genetics (1996) 14:457-60; Shalon et al., Genome Res. (1996) 6:639045; and Schena et al., Proc. Natl. Acad. Sci. USA (1992) 20:1679-84; and (iv) by dot-blotting on a nylon or nitrocellulose hybridization membrane (see, e.g., Sambrook et al., Eds. (2001) Molecular Cloning: A Laboratory Manual, 3rd ed., Vol. 1-3, Cold Spring Harbor Laboratory (Cold Spring Harbor, N.Y.)). Polynucleotides may also be noncovalently immobilized on the substrate by hybridization to anchors, by means of beads, or in a fluid phase such as in microtiter wells or capillaries. Arrays may include polynucleotide sequences prepared by the methods of invention.

For example, target-dependent ligation products may be prepared by the methods of the invention to include overlapping sequences of a viral genome, and such sequences immobilized on a solid support to generate an array. Such an array may be used to distinguish between viral strains by hybridization to specific subsets of sequences on the array.

In another aspect, libraries of the invention are used for development of diagnostic or forensic reagents for detection of the presence of bacterial and viral infectious agents, genetic traits and diseases, SNPs, etc. For example, libraries of the invention are used to select and optimize adjacent pairs of oligonucleotide probe sequences that are useful in ligase-mediated detection methods. In another example, libraries of the invention may be used to select and optimize polynucleotide sequences useful for hybridization-mediated DNA detection (i.e., affinity complementation). In a further example, libraries of the invention may be used to select and optimize polynucleotide primer sequences for PCR-based detection methods.

In another aspect, libraries of the invention may be used for development of affinity reagents. For example, a directed sequence library or a portion thereof, prepared by methods of the invention, may be coupled to a solid support and used for enrichment or purification of a polynucleotide sequence or nucleoprotein complex of interest from a mixture. Means for attachment of polynucleotides to a solid support are well known in the art. For example, amino-modified polynucleotides can be attached to an aldehyde-functionalized surface via reaction with free aldehyde groups using Schiff's base chemistry. In another example, amino-terminal polynucleotides can be coupled to isothiocyanate-activated glass, to aldehyde-activated glass, or to a glass surface modified with epoxide.

In other aspects, libraries of the invention may be used for preparative extraction of specific genes (including mRNA, genomic DNA, or fragments thereof), and as probes for specific sequences in Northern blots, in situ hybridization, and genomics mapping and annotation procedures.

In another aspect, libraries of the invention may be prepared from more than one target simultaneously (i.e., in a single reaction vessel). After cloning of directed sequence inserts obtained from multiple targets into vectors, the individual inserts may be sequenced and aligned to the appropriate target by, e.g., computer-assisted sequence alignment, to select desirable probe sequences for each target used in the mixture. These methods may be used to significantly enhance and accelerate genomics-related studies. Further, they can be used to generate cocktails of inhibitors of the expression of one or more genes, according to the targets used to generate the directed libraries. These cocktails can generated by expressing the libraries in cells of interest, selecting for a desired phenotype, and recovering the sequences of the library that conferred the phenotype by PCR and sequencing (see Li et al. (2000) supra; Kawasaki & Taira (2002), supra).

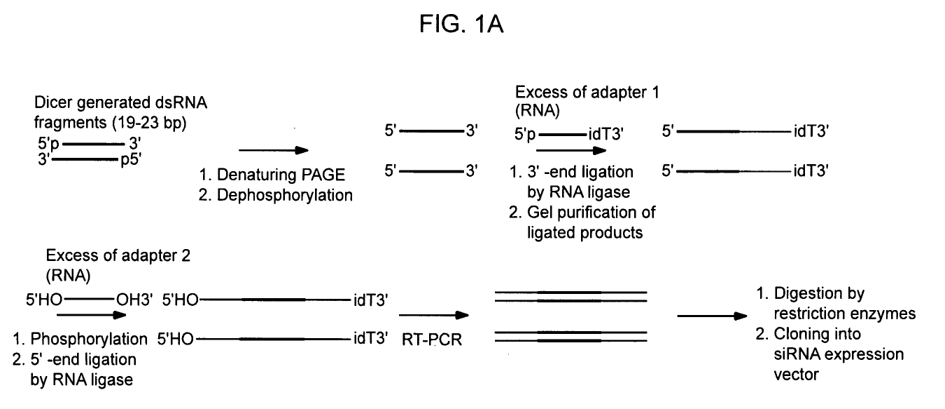

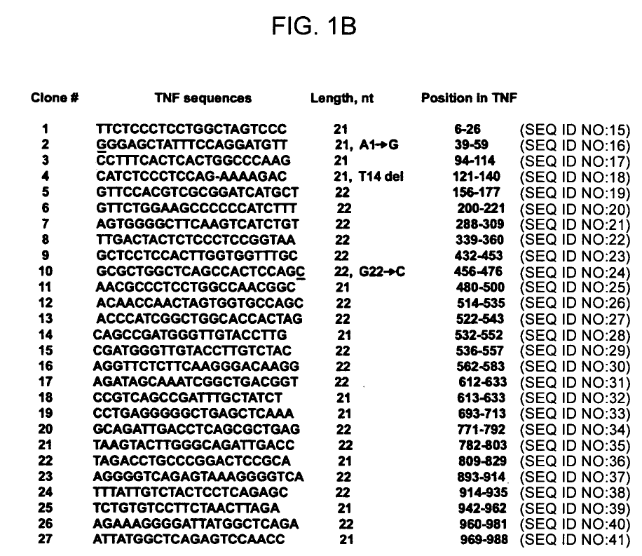

The scheme shown in FIG. 2, in contrast to the other schemes (FIGS. 1 and 3), typically yields several mismatches in the majority of the selected sequences; i.e., instead of a perfect directed library, an “enriched” library is produced. However, in addition to many of the above-listed uses, there are several potential applications for which the library of scheme 2 is especially suited. When it is desirable to identify a probe that distinguishes two closely related target sequences, such as alleles of a genetic locus, in some cases the best probe of a given length may have mismatches to both targets (Guo et al. 1997). Thus, a probe optimally discriminating between two alleles could be isolated by selecting from a library produced by the method of FIG. 2 for sequences that bind to one allele and further selecting the products of that screen against binding to the other allele.

Another use for the library of FIG. 2 is production of mutated sequences. The standard methods for introducing mutations include use of automated DNA synthesizers with nucleoside 3′-phosphoramidite solutions containing a small percentage of incorrect monomers, or alternatively “mutagenic” PCR. However, the enriched library obtained by the above-described method can be also utilized for this purpose.

Yet another potential application is selection of successful miRNA candidates from the obtained pool of mismatched sequences.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Production of a Directed Sequence Library for a TNF (Tumor Necrosis Factor-α) Target by the Dicer-Based Method

Transcription of the target. Sense and antisense strands of the RNA target were transcribed from a PCR-amplified DNA template either in one-tube reaction using opposing T7 promoters or separate-tube reactions, one using SP6, another T7 promoter (with Ambion's MEGAshortscript or MEGAscript kits).

Annealing and Dicer digest. RNA strands were annealed to form perfect duplex and digested by recombinant Dicer enzyme:

Dicer 6 μl (0.5 U/μl, Stratagene #240100-51)

5× buffer 6 μl

dsRNA+water 18 μl (˜3 μg)

Resulting 20-22 bp siRNAs were purified and strands-separated by 15% PAG-7M urea, eluted by crash/soak method and ethanol precipitated, then dissolved in 5 mM Tris-HCl pH 7.5.

The directed libraries produced by this method contain both sense and antisense gene-specific sequences. If it is desirable to obtain sequences that only correspond to the antisense strand, this library is mixed and annealed with an excess of antisense cDNA and the unhybridized antisense RNA fraction is separated by a gel-shift assay or affinity chromatography. However, this extra step is unnecessary for many purposes.

Dephosphorylation.

One potential problem of this approach is possible circularization via intramolecular ligation of the oligonucleotides during the ligation reaction. Therefore, the Dicer-produced RNA fragments are dephosphorylated, and in the first ligation reaction (see below) the flanking oligoribonucleotide 1 with a 5′-phosphate (required for ligation) has 3′-idT (inverted deoxythymidine) that prevents circularization.

fragmented RNA+water 85 μl

10× buffer 10 μl

CIAP 5 μl (Calf Intestine Alkaline Phosphatase, 1 U/μl, MBI Fermentas #EF0341)

The reaction proceeded for 1 h at 37° C., then followed phenol extraction, and RNA was precipitated with ethanol.

1^{st} Ligation.

Next, in two subsequent ligation steps, flanking oligoribonucleotides of defined sequences were attached to the 3′- and 5′-ends of each fragment by T4 RNA ligase:

T4 RNA ligase 1 μl (20 U/μl, NE BioLabs #M0204S)

RNase OUT 1 μl (40 U/μl, Invitrogen #10777-019)

0× buffer 4 μl

Flanking 1 oligo (5′-p; 3′-idT) 2 μl (150 pmol)

(SEQ. ID. NO. 1) (Sequence: 5′-GAGAAUMCAACAACAACAA-3′: Dharmacon, Lafayette, Colo.)

Fragmented RNA 1-10 μl (˜1 μg)

Water 31-22 μl

The reaction proceeded for 1 h at 37° C., the products were purified by 15% PAG-7M urea, and ethanol precipitated.

Phosphorylation

The gel-purified product of the 1st ligation was phosphorylated to be further ligated to another flanking oligoribonucleotide 2:

RNA+water 41 μl

10× buffer 5 μl

T4 PNK 2 μl (Polynucletide kinase, 10 U/μl, NE BioLabs #M0201S)

RNase OUT 1 μl

ATP 0.7 μl (75 mM)

The reaction proceeded for 1 h at 37° C., followed by phenol extraction and ethanol precipitation.

2nd Ligation

The phosphorylated product was ligated to flanking oligoribonucleotide 2, which does not have a terminal phosphate. In this second ligation reaction, the circularization of the product of the first ligation was also prevented due to the presence of 5′-blocking group.

T4 RNA ligase 1 μl

RNase OUT 1 μl

10× buffer 4 μl

Flanking 2 oligo 4 μl (300 pmol)

(SEQ. ID. NO. 2) (Sequence: 5′-UGGUACAUUACCUGGUAAC-3′)

RNA+water 30 μl

The reaction proceeded for 1 h at 37° C., followed by phenol extraction and ethanol precipitation.

Reverse Transcription

The products of 2nd ligation were reverse transcribed and further PCR amplified (RT-PCR) using the oligonucleotides attached to the gene-derived sequences as primer-binding sites.

5× buffer 10 μl

dNTPs 10 μl

RNA+water 26.5 μl

RT primer 0.5 μl (50 pmol)

AMV-RT 2 μl (10 U/μl, Promega #M510F)

RNase OUT 1 μl

The primers were annealed to RNA (65 C 5 min-ice), then other components were added and reaction incubated for 1 h at 42° C.

PCR Amplification.

10× buffer 10 μl

RT-DNA 10 μl (out of 50)

MgCl2 6 μl (25 mM)

dNTPs 8 μl (10 μl each/100 mM/+360 μl water)

RT primer 0.5-1 μl (50-100 pmol)

F primer 0.5-1 μl (50-100 pmol)

(Sequences: (SEQ. ID. NO. 3) 5′-TTGTTGTTGTTGTTATTCTC-3′ and (SEQ. ID. NO. 4) 5′-TGGTACATTACCTGGTAAC-3′: synthesized by IDT (Integrated DNA Technologies, Coralville, Iowa)

Taq 0.5 μl (Promega)

Water 64.5

Typical cycles (94° C. 30 sec—50° C. 30 sec—72° C. 30 sec) 10-20 cycles

Gel analysis. After PCR, 10 μl of the reaction mixture was mixed with 3 μl of 6× loading buffer (0.25% bromphenol blue, 0.25% xylene cyanol, 30% glycerol in water) and loaded onto a 10% native polyacrylamide gel in 1×TBE. The gel was run at room temperature at 25V/cm field. After electrophoresis, the gel was stained with ethidium bromide and visualized under UV light.

Cloning and Sequencing

The ˜60 bp products were PCR amplified on a large scale, gel purified, and cloned into the pT7Blue-3 vector (Novagen). E. coli were transformed with the recombinant vector and colonies were used for mini-preps. DNA was isolated using the QIAprep Spin Miniprep Kit (Qiagen), and sent to Retrogen, Inc. for unidirectional sequencing with T7 promoter primer.

Sequencing Results for Directed Library Against TNF Target

The sequencing results are shown in FIG. 1B. Of 27 sequences obtained for the TNF target, 24 had perfect match with and were evenly distributed along the target. 3 sequences contained single-nucleotide mismatches or deletions (indicated in bold), that are most likely explained by the multiple rounds of PCR using Taq polymerase. Higher fidelity thermostable polymerases (e.g. Pfu) could be used to fine tune the quality of the library sequences.

### Example 2

### Production of a Directed Sequence Library for a TNF Target by the Ligation-Based Method (Alternative #1)

DNA Target

The DNA target was a single-stranded murine TNFα cDNA. The target was prepared by amplification from a pGEM-4/TNF plasmid which included sequences for the murine TNFα gene with the full-length 5′-UTR and part of the 3′-UTR, totaling 1 kb. Amplification was by asymmetric PCR, using only a single primer, allowing production of single-stranded DNA. The single-stranded DNA was purified away from primers using a GeneClean III kit, ethanol precipitated, and used in experiments as a target for preparation of a directed library.

Hemi-Random Probes, Masking Oligonucleotides, and PCR Primers

Hemi-random probes, masking oligonucleotides, and PCR primers were synthesized by IDT (Integrated DNA Technologies, Coralville, Iowa).

Hemi-random probes contained 10-mer random regions and 26-mer defined sequences that contained a primer binding site and a restriction site, as follows:

Hemi-Random Probe A:

(SEQ. ID. NO. 5)

5′-pNNNNNNNNNNGGATCCCTGCTGACGACTAGACTGTG-3′
Hemi-Random Probe B:

(SEQ. ID. NO. 6)

5′-CAGTCTAGCAAGTATGCGTCCTCGAGNNNNNNNNNN-3′

Masking oligonucleotides contained sequences complementary to and masking the 26-nt long defined sequences of the probes. Masking oligonucleotides were used to prevent hybridization of the defined sequences of the probes to target sequences and to prevent parasitic ligation of probe sequences to each other. The sequences of the masking oligonucleotides were as follows:

Masking Oligonucleotide for Hemi-Random Probe A:
(SEQ. ID. NO. 7) 5′-CACAGTCTAGTCGTCAGCAGGGATCC-3′
Masking Oligonucleotide for Hemi-Random Probe B:
(SEQ. ID. NO. 8) 5′-CTCGAGGACGCATACTTGCTAGACTG-3′

Primers used for PCR amplification of ligation products were as follows:

(SEQ. ID. NO. 9)

Primer 1: 5′-CACAGTCTAGTCGTCAGCAG-3′

(SEQ. ID. NO. 10)

Primer 2: 5′-CAGTCTAGCAAGTATGCGTC-3′

Hybridization and Ligation

The hemi-random probes were pre-hybridized with their corresponding masking oligonucleotides in T4 DNA ligase reaction buffer for 5 min at room temperature. The target was added and the mixture was then incubated for 30 min at varying temperatures (25-42° C.) to allow the probes to hybridize to the target. T4 DNA ligase was then added and the mixture was incubated at room temperature for 1 hour. The ligation reaction mixture contained the following:

Hemi-Random Probes A and B 0.1-1 μM (2-20 pmol, 2-4 μl)

Masking Oligonucleotides for Hemi-Random Probes A and B 0.1-1 μM (2-20 pmol, 2-4μl)

DNA target 0.01-1 μM (0.2-20 pmol, 2 μl)

T4 DNA ligase buffer (30 mM Tris-HCl, pH 7.8, 5-10 mM MgCl12, 10 mM DTT, 1 mM ATP)

(2μl of 10×), 50-200 mM NaCl

T4 DNA ligase 0.1 U/μl (2 units, 1 μl)

H2O up to 20 μl

The effect of random oligodeoxyribonucleotides and oligoribonucleotides (4-5-6-7 nt long) and spermidine was also studied.

Amplification by PCR. After the ligation reaction was complete, 1 μl of the 20 μl ligation mixture was used for PCR amplification of the 72 bp ligation product. Typical cycles were: 94° C. 30 sec—54° C. 30 sec—72° C. 15 sec (20 cycles).

After PCR, 10 μl of the reaction mixture was mixed with 3 μl of 6× loading buffer (0.25% bromphenol blue, 0.25% xylene cyanol, 30% glycerol in water) and loaded onto a 10% native polyacrylamide gel in 1×TBE. The gel was run at room temperature at 25V/cm field. After electrophoresis, the gel was stained with ethidium bromide and visualized under UV light.

Cloning and Sequencing

The 72 bp ligation products were PCR amplified on a large scale, gel purified, and cloned into the pT7Blue-3 vector (Novagen). E. coli were transformed with the recombinant vector and colonies were used for mini-preps. DNA was isolated using the Wizard Plus Minipreps Purification System (Promega) or QIAprep Spin Miniprep Kit (Qiagen), and sent to Marshall University DNA Core Facility for dye-primer sequencing.

Sequencing Results for Directed Library Against TNF Target

The results of the target-dependent ligation experiments described above are shown in FIG. 2B.

### Example 3

### Production of a Directed Sequence Library for a DsRed Target by the DNase-Based Method (Alternative #2)

Preparation of gene-specific libraries by DNase I fragmentation of a dsDNA target (FIG. 3A)

PCR-amplified cDNA encoding DsRed was subjected to partial digestion with DNase I in a buffer containing 1 mM MnCl₂, 50 mM Tris-HCl (pH 7.5), 0.5 μg/μl BSA, and 0.1-0.3 U/μg DNase I (Ambion) at 20° C. for 1-10 min to generate small, blunt-ended DNA fragments (FIG. 2A). Under these conditions DNase I displays little sequence specificity, cleaving all regions of the DNA (except the terminal nucleotides) at an equal rate (Anderson 1981). Since DNase I generates fragments with a wide size distribution, reaction time and temperature were varied to determine optimal conditions to maximize the proportion of DNA in the desired size range (Anderson 1981; Matveeva et al., 1997). Aliquots were collected at various time points and quenched with an equal volume of loading buffer (95% formamide, 10 mM EDTA, 0.1% SDS) and DNA fragments corresponding to 20-30-bp were isolated by native 15% polyacrylamide gel. Next, nicks and potential gaps were repaired by T4 DNA ligase (MBI Fermentas) and DNA pol I (Klenow large fragment, MBI Fermentas) in 50 mM Tris-HCl (pH 7.5), 10 mM MgCl2, 0.1 mM NTPs, at 20° C. for 15 min.

The resulting DNA fragments (which contain 5′-phosphates) can be directly “blunt-end” cloned into the siRNA vector. However, attachment of adapters (fixed flanking double-stranded DNA sequences) is beneficial since it allows PCR amplification and higher ligation efficiency due to the presence of restriction sites in the adapters. The dsDNA adapters were essentially complementary to the 3′-termini of modified U6 and H1 promoters

(SEQ. ID. NO. 11)

5′-CTTGTGGAAAGAAGCTTAAAAAG;
Hi:

(SEQ. ID.NO. 12)

5′-AGTTCTGTATGAGACAGATCTAAAAAG).

Ligation reactions were performed with T4 DNA ligase, using one adapter at a time, each in ˜200-fold excess over the DNA fragments. The ligation products were PCR-amplified using primers complementary to the adapter sequences (94° C., 30 sec/52° C., 30 sec/72° C., 60 sec, for 20-30 cycles). The resulting ˜70 bp PCR products were purified by native 10% polyacrylamide gel, digested with Hind III and Bgl II, and after a second gel-purification, were cloned into the siRNA expression vector (see below). Plasmid DNAs isolated (QIAprep Spin Miniprep, Qiagen) from randomly selected bacterial clones were sequenced and used for transfection studies (FIG. 3B).

Sequencing Results for the Directed Library Against DsRed

Sequencing of several clones obtained from this approach showed that all the isolated clones contained inserts that had perfect homology to the DsRed gene. DsRed insert sequences varied from ˜17 to 34 bp (FIG. 3B). Although a few shorter (17 bp) and longer (34 bp) inserts were obtained, more than half of the inserts were 19-25 bp in size and distributed fairly uniformly throughout the DsRed gene, indicating that no portion of the sequence was highly over- or under-represented in the limited number of clones examined.

### Example 4

### Selection of Optimal Tarqet Sequences with a TNF-Directed Lasso Library Produced by the Dicer-Based Method

In vitro Selection Protocol

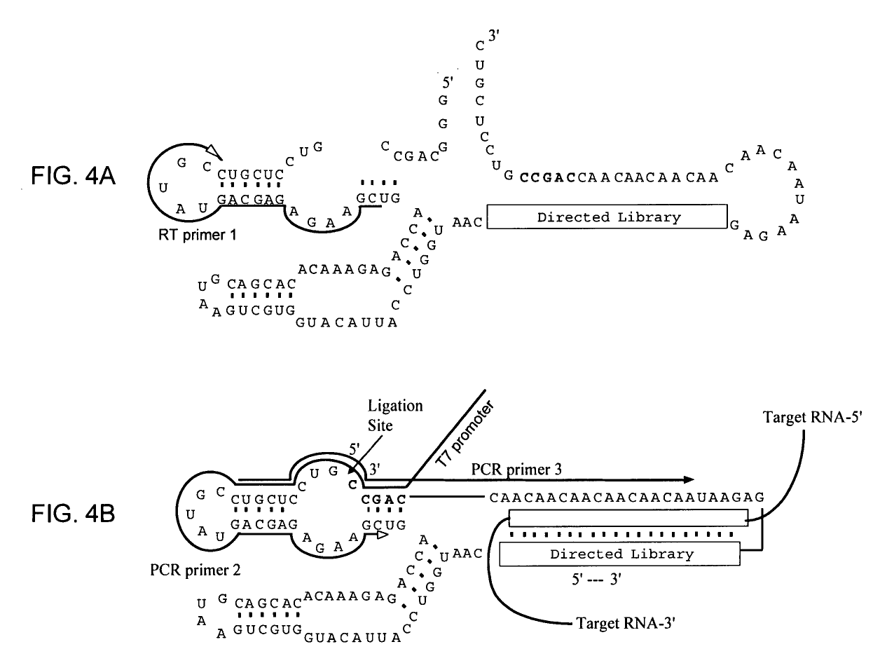

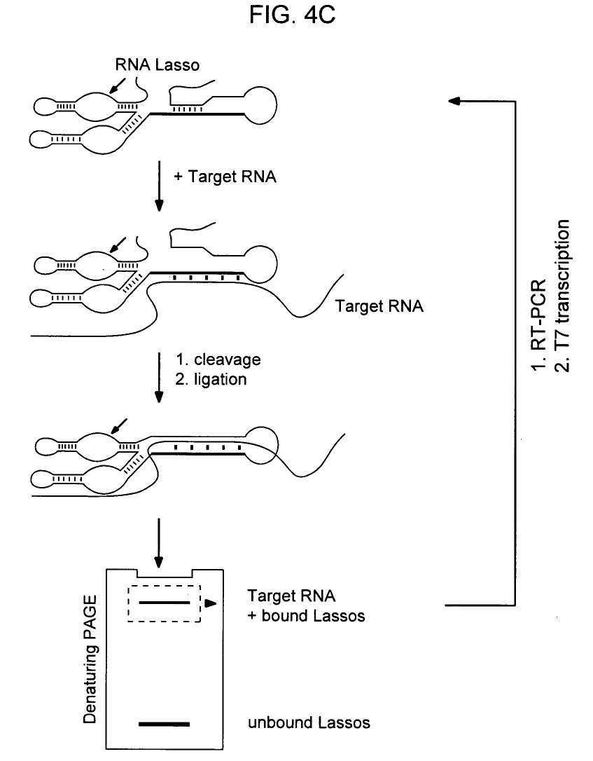

A TNF-directed Lasso library generated as described in Example 1 was transcribed in vitro with T7 RNA polymerase (Ambion) to generate the initial pool of Lassos for in vitro selection (FIG. 4A). We confirmed that the transcribed library contains active Lasso species that can self-process and circularize. Three rounds of selection were performed with primers for RCA-RT-PCR as depicted in FIGS. 4A-B. For the initial round of selection, 400 pmol of the Lasso directed library was incubated with an excess of target TNF-1000 RNA at 37° C. for 60 min in SB buffer. These conditions ensure that the library complexity is retained through the initial round of selection. Reactions were electrophoresed on a denaturing 6% polyacrylamide gel to separate free Lasso and free target RNA from the Lasso-target complex (see FIG. 4C). RNA was visualized in the gel by ethidium bromide staining, and the appropriate gel slices were excised and complexes eluted before amplifying by RCA-RT-PCR as described above. The RT-PCR product was gel purified on a 1.5% agarose gel and extracted using QIAquick Gel Extraction Kit (Qiagen). The resulting DNA was used as the transcription template to generate the enriched Lasso library for the next round of selection. The entire selection process was repeated twice with decreases in incubation time (30 min for round 2, and 5 min for round 3).

Results of the in vitro Selection

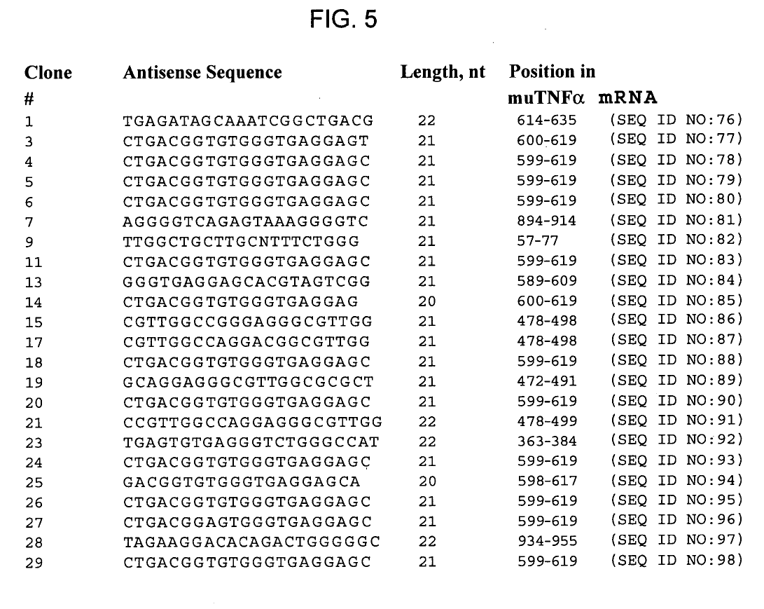

After the third round of selection, the gel-purified RT-PCR fragment was cloned using a TA-cloning kit (Invitrogen). The resulting colonies were screened for inserts by blue/white color selection. 23 individual clones were isolated and sequenced to identify the selected antisense sequences (FIG. 5). As expected from the directed library synthesis, the target sequences range from 20-22 nucleotides, consistent with the length of the gene-specific fragments in the directed library (see above). The few mismatches observed are indicated in lowercase. 14 of the 23 sequences clustered in the region between nucleotides 589 and 619 (indicated by *). Four clones were identified with sequence surrounding nucleotides 472-499. All other sites were represented by one clone.

Analysis of Individual Selected Lassos

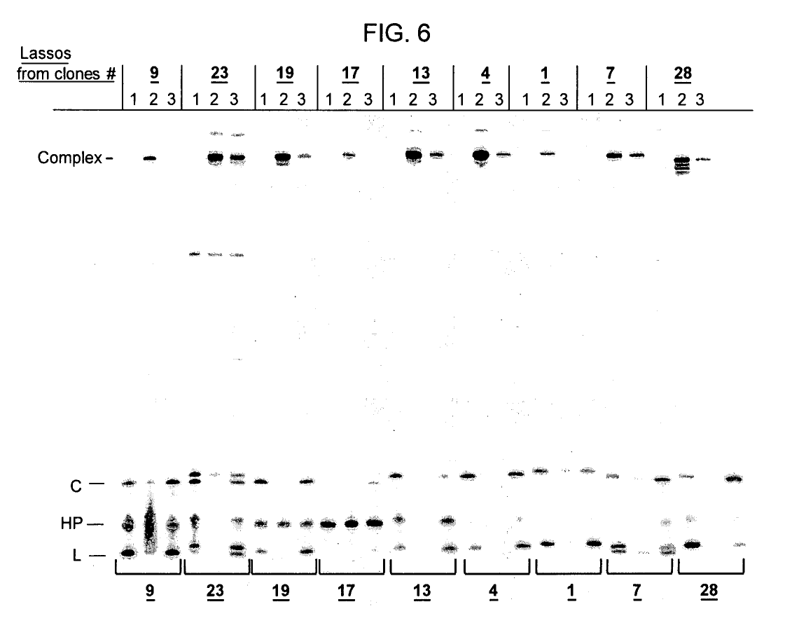

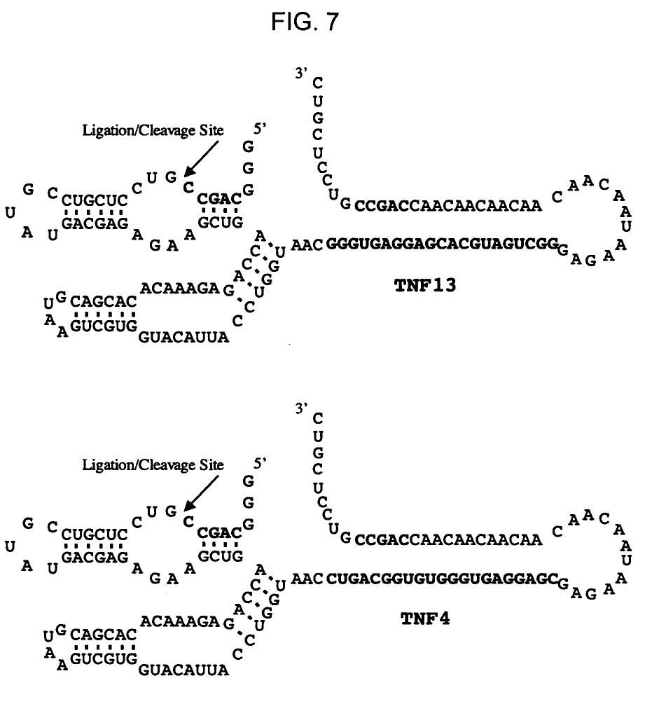

To identify which of the selected Lassos are superior binders, one representative clone of each unique selected sequence Was transcribed in vitro and tested in binding affinity and kinetics assays. Lassos were internally ³²P-labeled during in vitro transcription and incubated with an excess of non-radioactive target TNF-1000 RNA at 37° C. in SB. Products of these reactions were analyzed by denaturing 5% PAGE (FIG. 6). From this additional screen, #13 and #4 were identified as two of the strongest and fastest binders (FIG. 7). Both of these sequences, which bind sites 10 nt apart, target the most represented site of TNFα that was identified in this selection (spanning the 589-619 nts site).

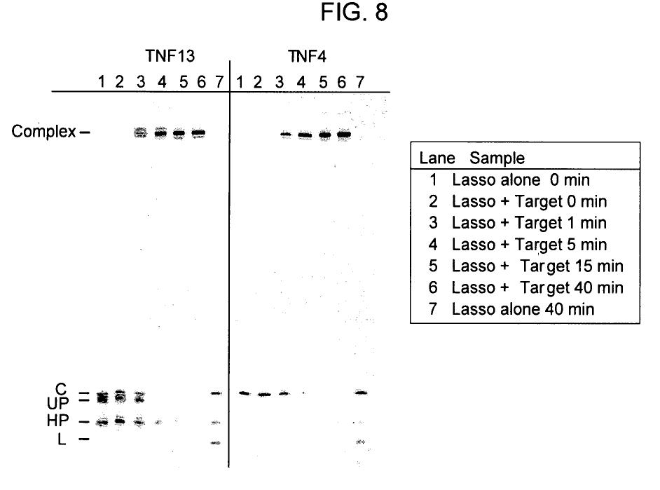

Lassos were synthesized and internally radiolabeled by T7 polymerase transcription in the presence of [α³²P]rCTP. Time course binding assays were performed to monitor the efficiency of Lasso binding to target RNA (FIG. 8) for Lassos #13 and #4. Both are completely bound within five minutes of incubation with target RNA.

In conclusion, by starting with a pool of Lassos that contain a gene-specific library against mTNFα, we were able to select the most efficiently hybridizing and circularizing Lassos. We confirmed that the Lassos selected were capable of fast binding to target RNA by testing the selected sequences individually in binding assays.

### Example 5

### Selection of Optimal Target Sequences with a DsRed-Directed Lasso Library Produced by Dicer-Based Method

Selection for optimal DsRed target sequences was performed essentially as described for TNFα. After three rounds of selection, the resulting Lassos were cloned and sequenced to determine which antisense sequences were selected.

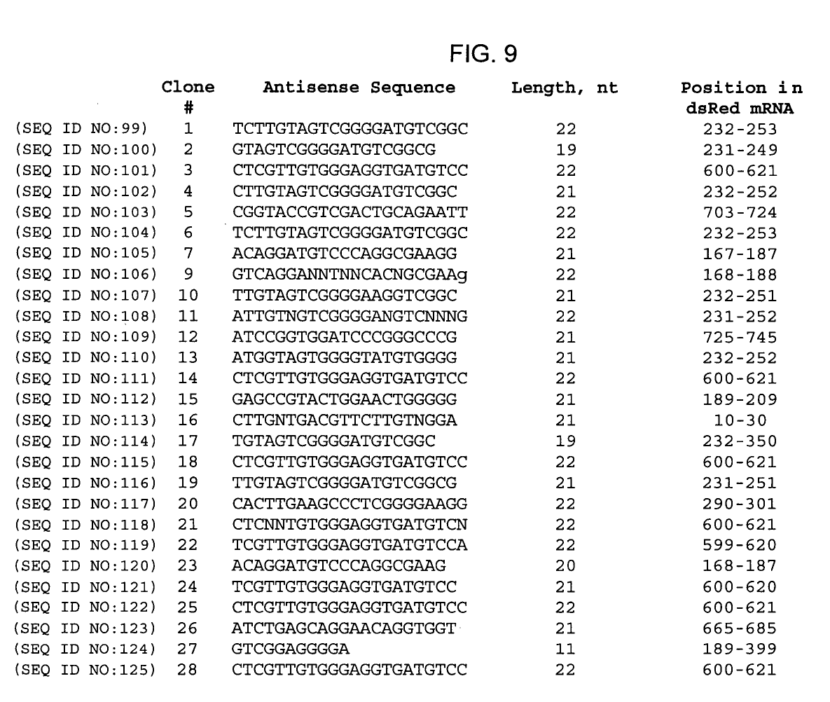

Results are shown in FIG. 9.

### Example 6

### shRNA Library Generation Strategy #1

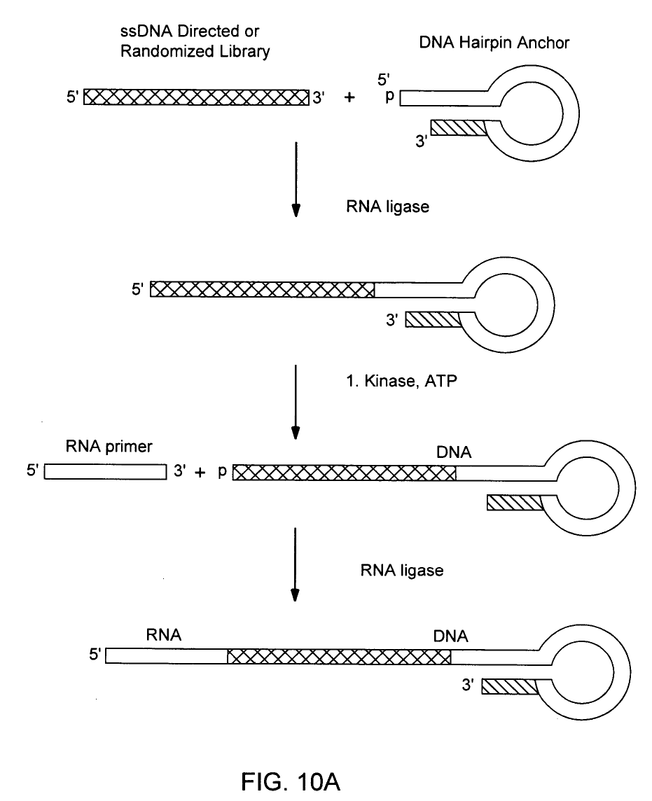

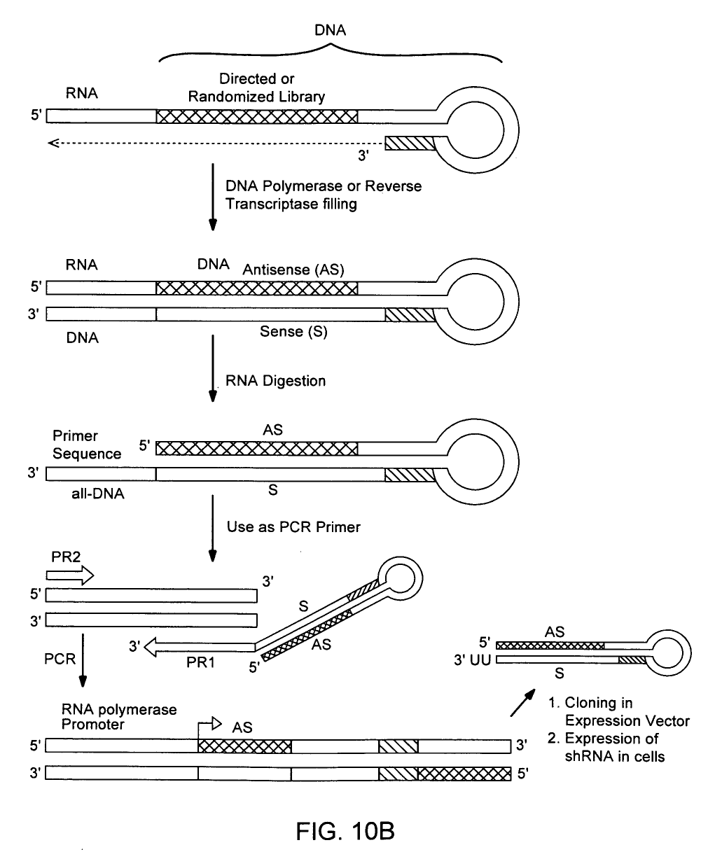

The directed or randomized oligonucleotide libraries within desirable length range, obtained as shown in FIGS. 1-3 or by any other method known in the art (e.g., oligonucleotide synthesis or chemical and/or enzymatic fragmentation of cDNA), can be incorporated into an shRNA expression cassette template using RNA ligase as shown in FIG. 10A. The ssDNA oligodeoxyribonucleotides from the libraries are ligated first to a DNA hairpin at the 3′-end and then to a ssRNA at the 5′-end, producing an RNA-DNA chimera. The DNA hairpin can be of any desired sequence but must have a non-palindromic 5′ overhang of a few nucleotides, terminating in a 5′-phosphate. The overhang both increases the efficiency of intermolecular ligation by RNA ligase and prevents circularization of the hairpin. After ligating the DNA library oligonucleotides to the DNA hairpin, the 5′-end of resulting DNA is phosphorylated by polynucleotide kinase and ligated to the 3′-end of the ssRNA, which encodes an antisense PCR primer sequence. In the next step (FIG. 10B) the 3′ end of this RNA-DNA chimera is extended by a fill-in reaction using any DNA polymerase capable of using either DNA or RNA as a template. The resulting RNA-DNA hairpin then is treated by any agent that can specifically hydrolyze (or cleave through a transesterification reaction) the RNA but preserve the DNA, such as ribonucleases or metal ions or alkali. The resulting DNA-only hairpin molecules have a 3′-end overhang that can serve as a PCR primer in a synthetic amplification reaction to attach a promoter (e.g., U6 or H1, or pol II), similar to the reaction previously described for preparation of defined sequence shRNA expression cassettes by Scherer et al. (2004) Method 10: 597-603.

This shRNA PCR transcription cassette can be used either directly for transfections of mammalian cells or after cloning into appropriate expression vectors. A direct transfection system can be used for rapid screening of siRNA libraries and allows easy identification of optimal siRNA-target sequence combinations and multiplexing of siRNA library expression in mammalian cells. This strategy also avoids a bacterial amplification stage, which can introduce major mutations or deletions at inverted repeats. Note that 5′-phosphorylation of the primers results in enhanced expression of PCR cassettes, probably stabilizing them in cells. Alternatively, this cassette can be capped with hairpin forming oligodeoxynucleotides. This approach was shown to stabilize by protecting the termini of the DNA duplex from exonucleolytic degradation resulting in improved expression in cells (Horie & Simada, 1994, Biochem. Mol. Biol. Int.)

Alternatively, dsDNA templates for the directed siRNA library can be generated by using DNase I, dicer or ligation methods. The DNA duplex is then digested with restriction enzymes Hind III and Bgl II generating overhangs immediately next to the randomized sequence. A hairpin-shaped oligonucleotide containing H1 or any other pol III promoter sequence and having a Bgl II restriction site at the end of the stem is ligated to the 3′-end of the duplex DNA, converting the duplex into a hairpin. A second set of synthetic dsDNA (PR1 and PR2) with Hind III restriction site at its 3′-end is ligated to the above siRNA-H1 hairpin product. The resulting DNA hairpins with a 3′-end single stranded overhang having homology to the U6 promoter are gel-purified under denaturing conditions, and then used as reverse primers in the PCR reaction on a hU6 promoter plasmid as template as described above and as shown in FIG. 10B.

### Example 7

### Alternative Library Approach: TA Cloning Scheme

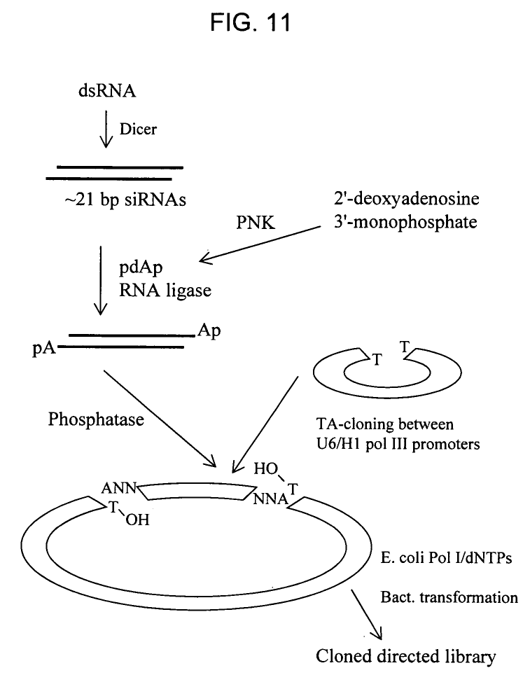

Double-stranded RNA corresponding to the target of interest is prepared and cleaved with recombinant dicer enzyme as described above. The diced ds RNA fragments (approximately 21 bp with 2 nt 3′ overhangs) are treated with calf intestinal phosphatase and the 5′ dephosphorylated dsRNA is purified by phenol/chloroform extraction and ethanol precipitation (FIG. 11). Next, 2′-deoxyadenosine 3′ monophosphate is treated with polynucleotide kinase and the resulting pdAp is ligated to the dsRNA fragments using RNA ligase. Following ligation, the ligase is inactivated by heating to 65 C, the fragment 5′ end dephosphorylated with calf intestinal phosphatase, and the purified fragment is ligated into a linearized opposing PollII promoter expression vector containing a 3′ deoxythymidine overhang. The gaps in the ligated vector (cause by the original 2 nt 3′ overhangs on the 21 bp dsRNA fragments) are filled in with E. coli Poll in the presence of dATP, dGTP, dCTP and dTTP. The plasmid library containing the dsRNA inserts is then transformed into competent bacteria to amplify the library species.

### Example 8

### shRNA Library Generation Strategy #2

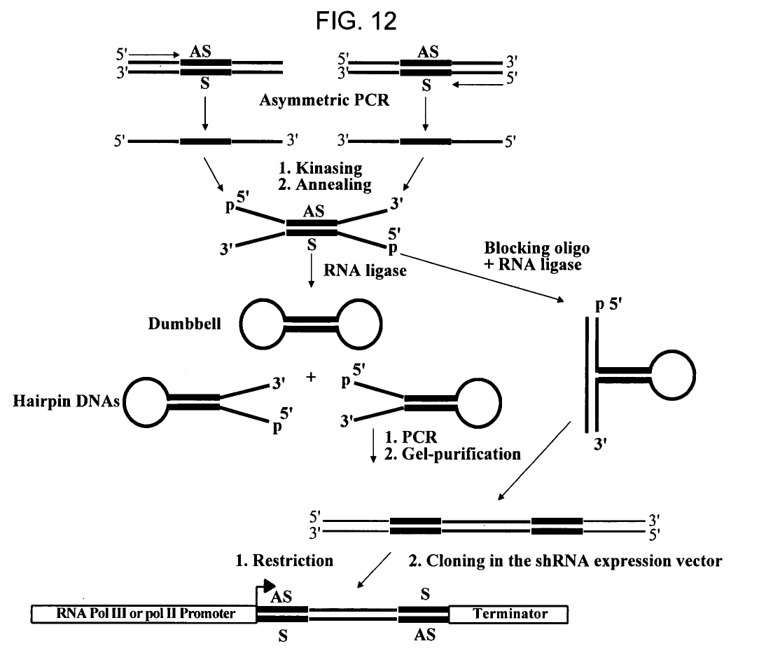

Two dsDNA directed libraries, generated by one of the methods shown in FIGS. 1-3, which have the same pool of gene-specific antisense (AS) and sense (S) sequences but differ in the arrangement of the flanking primer sequences as shown in FIG. 12, are converted into two pools of ssDNA oligonucleotides by asymmetric PCR. The pools are phosphorylated at their 5′ ends, mixed together, denatured, and annealed to achieve cross-hybridization. By this procedure, DNA-DNA complexes having both fully complementary AS/S duplexes as well as non-complementary overhangs at both ends are formed. Ligation of these overhangs by RNA ligase yields a mixture of hairpin and dumbbell-shaped DNAs as shown in FIG. 12. Blocking oligonucleotides that are complementary to either of the two types of overhangs can direct the ligation reaction toward formation of only hairpin structures. These DNA hairpins are then amplified by PCR by the hairpin amplification procedure described in (Kaur and Makrigiorgos (2003) NucI Acids Res. 31: e26). The resulting dsDNA fragments encoding shRNA libraries can be cloned into a pol IlIl (or pol 11) expression vector for expression of the shRNA library in cells.

### Example 9

### Conversion of siRNA Library Encoding dsDNA Fragments Generated by Enzymatic Fragmentation into Inverted Repeat Cassettes for Transcription of shRNAs

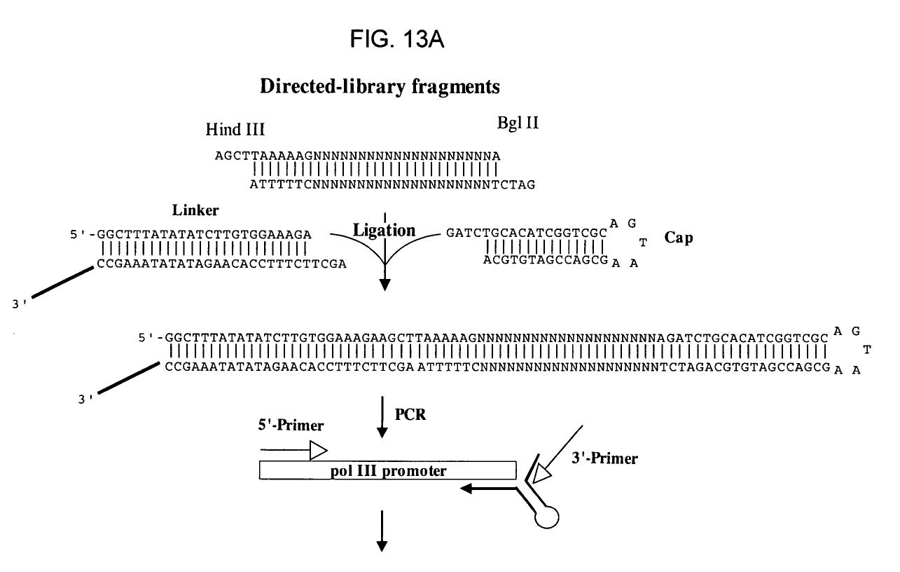

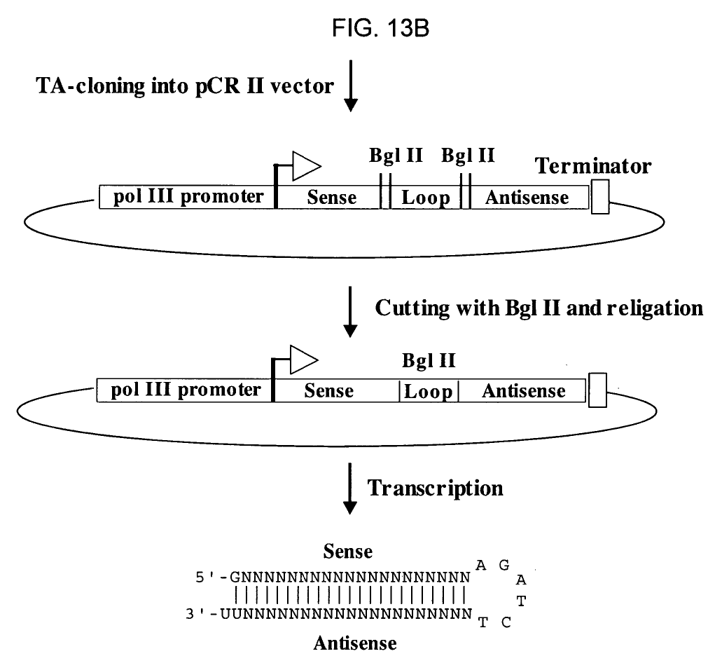

The directed library (obtained by any method described above), is digested with Hind III and Bgl II and ligated to two linkers, one in the form of a hairpin (CAP) and the other a partial duplex DNA containing a 3′-tail that is complementary to the 3′-end of the h-U6 promoter (FIG. 13). This product is then used as a reverse primer alongside a primer specific to 5′-end of the U6 promoter resulting in a U6 transcription cassette. During the PCR reaction this hairpin DNA with a 3′-overhang complementary to the 3′-end of the human U6 promoter acts as a reverse primer incorporating the inverted sequence feature to the 3′-end of the U6 promoter. The PCR product is ligated into pCRII vector. Plasmids are then digested with Bgl II to remove the extraneous sequences flanking the loop and religated, forming the final product, expression-ready shRNA vectors. The transcribed shRNA is shown at the bottom.

### Example 10

### Expression of High Copy shRNA Libraries from Multimeric H1-shRNA Cassettes

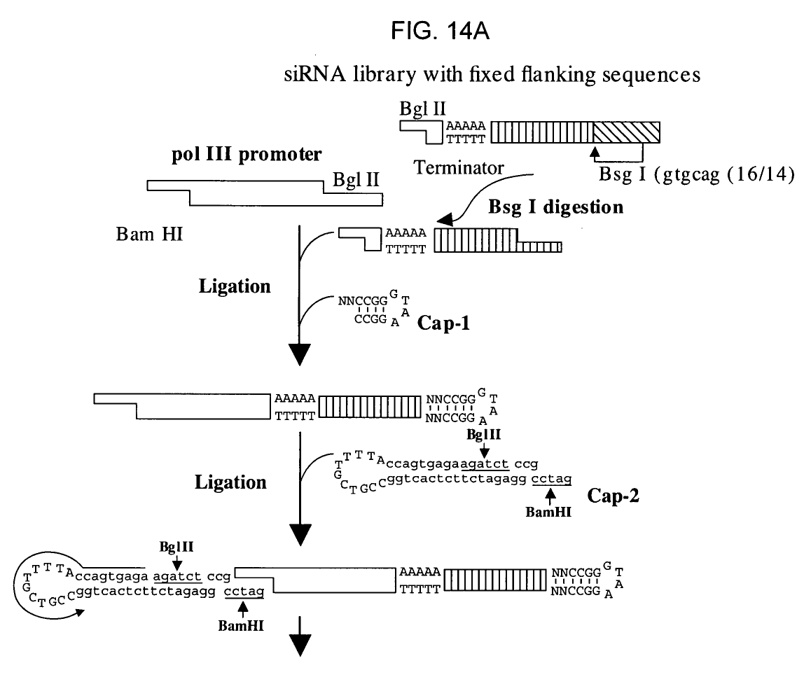

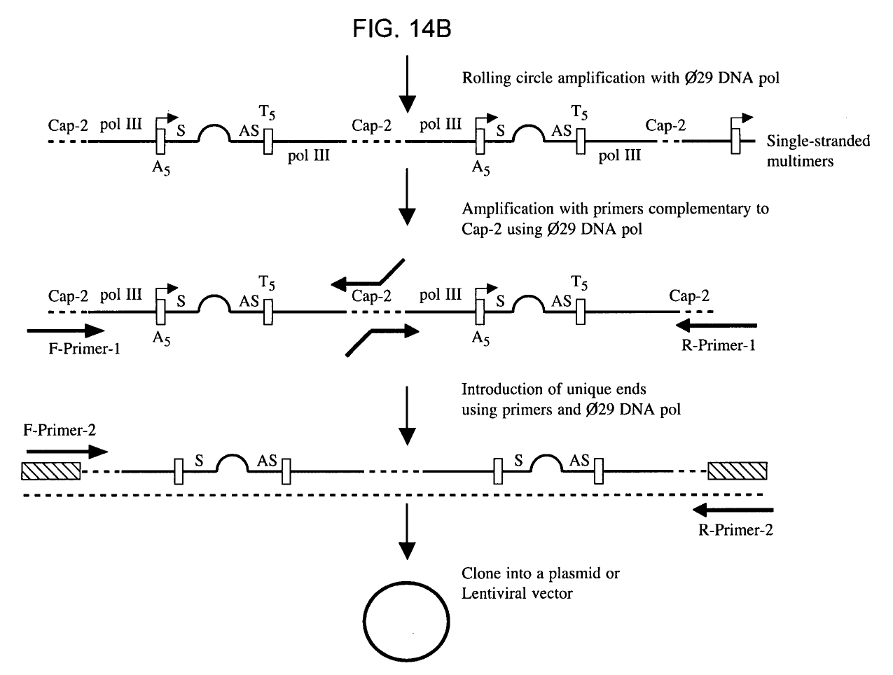

The goal: to convert the fused product between pol III (U6 or H1) promoter and restriction fragment, encoding a directed siRNA library, into a dumbbell-shaped DNA follwed by its RCA amplification. To generate multimeric pol III promoter-shRNA cassettes by RCA reaction using Ø29 (Blau, 04) or with Bst I DNA pol. (Shirane et al., 04) pol (FIG. 14) and convert concatemeric ssDNAs into dsDNA by using flanking primers containing primer binding sequences. These primers will be complementary to 5-and 3′-end of the H1 promoter. Upon annealing the first primer, ssDNA is extended producing a strand complementary to the 5′-unique end of the primer. Same fill-in reaction is performed with the 5′-specific primer which also contain a unique primer binding site. These unique sequences are used as primer binding sites in the subsequent PCR reaction. Alternatively, linkers with unique sequences can be attached and used as primer binding sites.

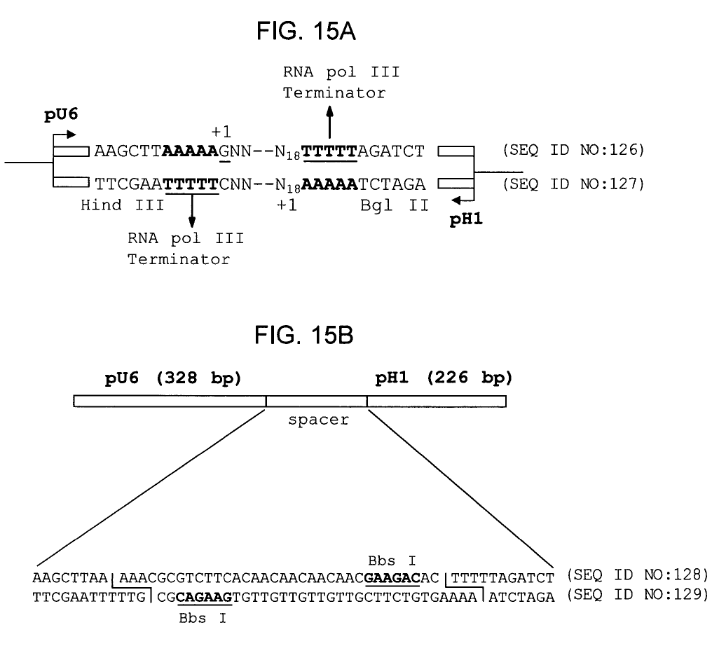

Improved method for expression of directed libraries of shRNAs: In this method (FIG. 14), the directed library in DNA form is generated by one of the methods of FIGS. 1-3, with flanking sequences containing oligo dA/oligo dT (as pol III transcriptional terminator) on one side and a Bsg I restriction site (for cutting within the variable sequence) on the other. This library of fragments is ligated to a pol III promoter such as H1, such that the transcriptional terminator sequence replaces an equivalent number of base pairs of between the TATA box and the 3′ end of the H1 promoter (FIG. 15) (Zheng et al., PNAS 101, 134 [2004]). Following Bsg I cleavage, a stem-loop “cap” sequence is ligated on the end opposite the H1 promoter and a second stem-loop cap is ligated on the 5′ end of the H1 promoter after cleavage of the terminal sequence to produce “sticky ends.” The resulting dumbbell-shaped, circular molecule is subjected to rolling circle amplification (RCA) using a primer as shown in FIG. 14, generating multimeric linear molecules which, after second strand synthesis and transcription with pol III, generate RNAs that terminate immediately after the target-specific sequence and fold into shRNAs (Sen et al., Nature Genetics 36, 183 [2004]). The RCA step provides for increased numbers of copies from each separate library sequence and also expresses shRNAs from convergent pol III promoters. If expressed using a lentiviral or other integrating vector, with one or at most a few copies integrated per cell, each cell would express many copies of a single library sequence, allowing for more efficient selection of individual sequences since each sequence would be strongly expressed.

### Example 11

### Inhibition of TNF by siRNAs (from a TNF-Directed Library) and shRNAs (Rationally-Designed) Expressed from Opposing or Unidirectional-Promoter Vectors

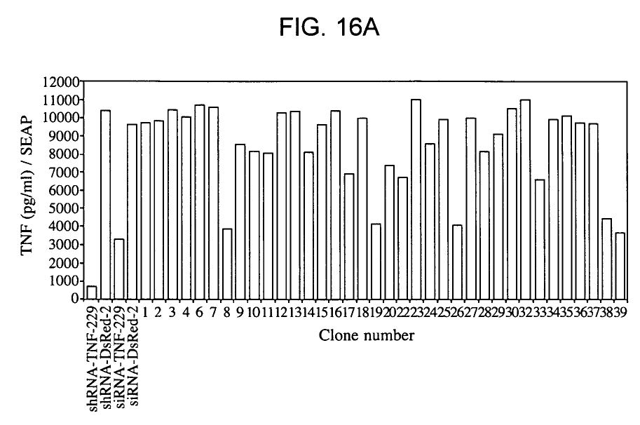

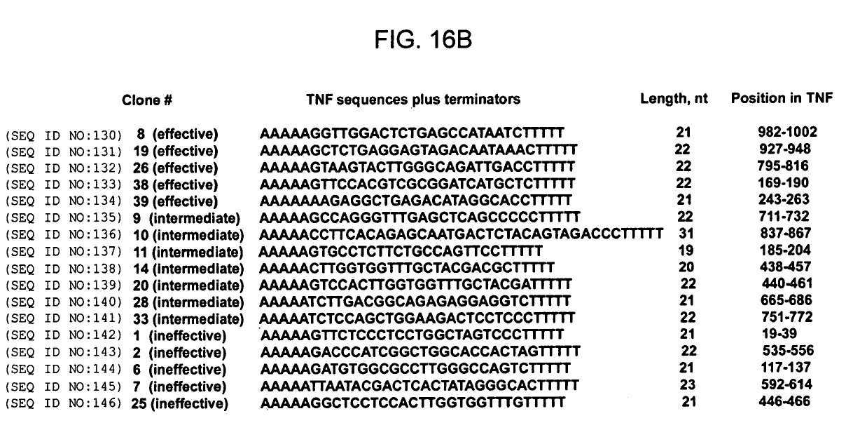

The experimental design of the constructs and experimental scheme is shown in FIG. 15A-B. TNF expression vector was cotransfected with the indicated pol III shRNA inhibitor and and pSEAP [secreted alkaline phosphatase (SEAP) to control for transfection efficiency] expression vectors into 293FT cells with lipofectamine 2000 (Invitrogen). Supernatants were collected 62 h after transfection, diluted and and were assayed by ELISA for TNF and SEAP (supernatants for SEAP were collected at 48 h post-transfection) assay for secreted alkaline phosphatase and the results were presented as pg/ml TNF/SEAP or pg/ml TNF and SEAP. Several clones that showed inhibitory effect were also sequenced. Opposing pol III promoter constructs encode 21-nt fixed sequence control siRNAs (U6/H1 (S)DsRed and TNF 229) and 21-22-nt DsRed-directed library siRNA sequences. The fixed-sequence shRNAs vector (DsRed-2) contained a 29 nt stem and a miRNA 23 loop sequence (SEQ. ID. NO. 13) (CUUCCUGUCA) to aid cytoplasmic localization. The results are shown in FIG. 16.

### Example 12

### Inhibition of DsRed Expression by siRNAs (DsRed-Directed Library Sequences Obtained by DNase Method and Rationally-Designed Fixed) or Small Hairpin Expressed from Opposinq pol III Promoters in Transiently Transfected 293 Cells

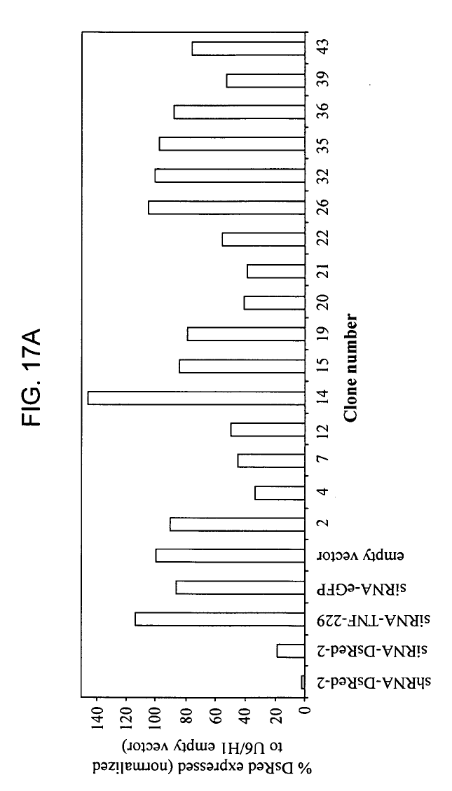

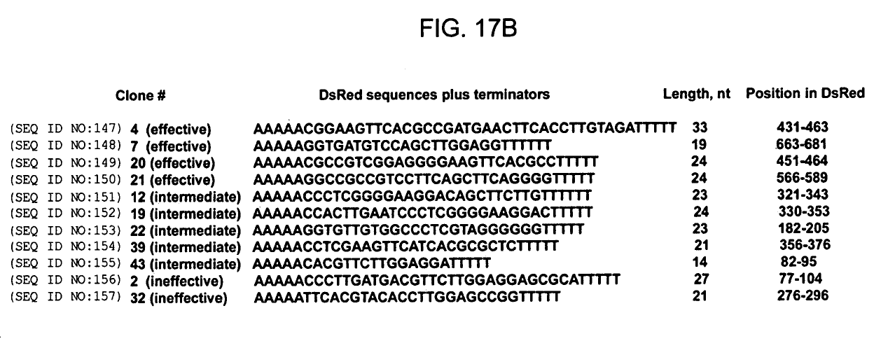

The experimental design of the constructs is shown in FIG. 15A-B. DsRed expression vector was cotransfected with the indicated pol III shRNA inhibitor expression vectors into 293FT cells with lipofectamine 2000 (Invitrogen). Cells were imaged by fluorescence microscopy and analyzed by flow cytometry 36 hours after transfection. The amount of inhibition of each siRNA was normalized to U6/H1 (S) empty vector. Opposing pol III promoter constructs encode 21-nt fixed sequence control siRNAs (U6/H1 (S)DsRed, eGFP, and TNF 229) and 19 to ˜27 nt DsRed-directed library siRNA sequences. The fixed-sequence shRNAs vector (DsRed-2) contained a 29 nt stem and a miRNA 23 loop sequence (SEQ. ID. NO. 14) (CUUCCUGUCA) to aid cytoplasmic localization. The results are shown in FIG. 17.

### Example 13

### Selection of Antiviral Inhibitors from RNA Libraries in Cultured Cells

Here we describe a rapid, automatic, in vivo method for identifying the best target genes in a virus and the most accessible target sequences within those genes. The scheme for this approach is summarized in FIG. 16. The method involves generating cell lines expressing directed libraries of RNA inhibitors and challenging them with the virus of interest. Cells that survive the infection are recovered and analyzed for the sequence of RNA inhibitors that apparently conferred resistance. The sequence of the antisense component of the RNA inhibitor reveals the target gene(s) whose inhibition prevented viral cytotoxicity. It also reveals a sequence of that target gene that is accessible to antisense disruption as well as the sequence of the RNA molecule that is an effective inhibitor. These target mRNA sequences should be accessible for attack by any RNA-targeting technique, whether it be antisense, ribozyme, RNAi, or Lasso. This information is validated by synthesizing the identified RNA inhibitors de novo and testing for their ability to confer resistance to the virus.

A unique feature of this approach is that the selection takes place within the cell, and directed libraries containing only target-specific molecules are employed. The complexity of the viral or cDNA directed library is relatively small, on the order of 10⁴ for the most viral RNA targets and 10-20×10⁶ for cDNA. This allows establishment of the antisense library in host cells with little or no loss of complexity.

The initial experiments are carried out with a non-replicative form of SFV (SQL), which cannot propagate unless it has been treated with protease.

Once putative inhibitors are identified, they are tested individually for efficacy, specificity and potency with chymotrypsin-treated SQL SFV virus and finally with the fully virulent replication proficient A7 strain. An eventual goal is to develop a panel of cell-based libraries that will allow infection with a wide variety of viral pathogens to screen for inhibitors.

To deliver the RNA inhibitors, lentiviral vectors are used. These vectors deliver transgenes very efficiently to many primary cell types. The use of strong pol III promoters (U6, tRNA or H1) in these vectors assures high levels of intracellular expression of RNA inhibitors. If even higher expression levels are needed, an enhanced U6 promoter recently reported can be used.

### Example 14

### Selection Scheme Using HSV Thymidine Kinase and Ganciclovir

In this example (FIG. 17), protection from drug-induced cell death is used as a surrogate for protection from viral cell killing. Specifically, stable cell lines are generated, expressing a recombinant mRNA containing DsRed (similar to green fluorescence protein), HSV thymidine kinase (TK), and a target of interest. These cells are infected by a recombinant lentivirus expressing a library of inhibitors. Addition of the purine nucleoside analog drug, ganciclovir, causes killing of all cells expressing the TK fusion protein. Cells expressing, for example, a Lasso that blocks translation of the DsRed-TK-viral mRNA, or an siRNA that causes degradation of the mRNA, are rescued from killing by ganciclovir. RNA from these cells is analyzed to determine the sequence of the protective siRNA, which reveals the identity of the target whose inhibition was protective. The final aspect is to test the ability of the candidate inhibitors to block infectious viral propagation in cell lines.

Targeting Host Cellular Factors:

The ability of siRNAs to inhibit viral replication has been shown for several pathogenic viruses; however, considering the high sequence specificity of siRNAs and high mutation rates of RNA viruses including SFV, HCV, HIV and poliovirus, the antiviral efficacy of siRNAs directed to the viral genome may be limited due to the potential emergence of escape mutants. However, cellular factors involved in the viral life cycle have been successfully targeted providing a more sustained siRNA effect since these factors do not normally mutate and are present at much lower copy number than the viral RNA targets. For example, targeting of HIV's main receptor CD4, its coreceptor, CCR5, or both CCR5 and CXCR4, can suppress the entry and replication of HIV-1. Since viral entry and replication require various host factors, an siRNA library generated using a host cDNA library alongside an HIV-directed siRNA library can be used to identify several host and viral targets essential for viral infection.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. A method of producing a target-specific library that comprises substantially all sequences of a pre-determined length or range of lengths that are comprised within a target polynucleotide sequence, the method comprising:
digesting a double-stranded RNA copy of said target polynucleotide with a nuclease to generate fragments of from about 10 nucleotides to about 40 nucleotides in length;
dephosphorylating said RNA fragments;
ligating said RNA fragment to a first flanking oligonucleotide comprising a 3′ terminator nucleotide to generate a first ligation product;
phosphorylating said first ligation product;
ligating to said first ligation product a second flanking oligonucleotide lacking a 5′ phosphate group to generate a second ligation product; and
reverse transcribing said second amplification product to generate a cDNA;
amplifying said cDNA with primers complementary to said first and said second flanking oligonucleotide;
wherein said resulting library of polynucleotides comprises substantially all sequences of a pre-determined length within said target polynucleotide sequence.

2. A method of producing a target-specific library that comprises substantially all sequences of a pre-determined length or range of lengths that are comprised within a target polynucleotide sequence, the method comprising:
digesting a double-stranded RNA copy of said target polynucleotide with a nuclease to generate fragments of from about 10 nucleotides to about 40 nucleotides in length;
dephosphorylating said RNA fragments;
ligating 2′-deoxyadenosine 3′-monophosphate (pdAp) to each end of said product of dephosphorylation;
dephosphorylating the product of said ligation reaction;
ligating product of said dephosphorylation reaction into a linearized vector having 3′-deoxythymidine overhangs;
filling in gaps by using a DNA polymerase such as E. coli Pol l;
amplifying the resulting vector in bacteria to replace RNA with DNA;
wherein said resulting library of polynucleotides comprises substantially all sequences of a pre-determined length within said target polynucleotide sequence.

3. The method according to claim 1, further comprising the step of strand-separating said double stranded RNA fragments to provide single stranded RNA fragments.

4. The method of claim 1 wherein said double-stranded RNA copy of said target polynucleotide is generated by transcription of DNA templates.

5. The method of claim 2 wherein said double-stranded RNA copy of said target polynucleotide is generated by transcription of DNA templates.

6. The method according to claim 1, wherein said nuclease is a length-directed RNAse.

7. The method according to claim 2, wherein said nuclease is a length-directed RNAse.

8. The method of claim 6, wherein said length-directed RNAse is a member of the RNAse III family.

9. The method of claim 6, wherein said length-directed RNAse is Dicer and said fragments or from about 17 to 27 nucleotides in length.

10. The method of claim 6, wherein said length-directed RNAse is ExoIII and said fragments are from about 10 to about 30 nucleotides in length.

11. The method of claim 3, wherein said strand separating step is performed by heat-denaturation.

12. The method of claim 1, wherein said dephosphorylating step is carried out with calf intestinal phosphatase.

13. The method of claim 1 wherein at least one of said first or said second flanking oligonucleotide comprises a recognition site for a restriction endonuclease.

14. The method according to claim 13, further comprising at least one of the steps of:
digesting said library of polynucleotides with a restriction endonuclease that cleaves in the ligated flanking sequences.

15. The method of claim 1, further comprising the step of inserting library into a vector.

16. A method of producing a target-specific library that comprises substantially all sequences of a pre-determined range of lengths that are comprised within a target polynucleotide sequence, the method comprising:
partially digesting a double-stranded DNA copy of said target polynucleotide with DNase I, and digestion is performed in the presence of Mn⁺² to generate blunt-ended fragments of from about 10 nucleotides to about 40 nucleotides in length or a wider range that comprises the range 10 to 40 nucleotides; and
ligating said DNA fragment to a first adapter;
ligating the above product to a second DNA adapter.
amplifying the product of the above reaction using primers complementary to said first and said second adapters.
inserting said fragments into a vector or between fixed sequence segments of DNA.

17. The method of claim 16, wherein at least one of said first and second primers contain a restriction site.

18. The method according to claim 16, further comprising the steps of purifying the product of the ligation after ligating to said first primer, and before ligating to said second primer.

19. The method according to claims 17, further comprising the steps of:
digesting the product of ligation or amplification with one or two restriction endonucleases targeted to a sequence in one or both adapters.

20. A method of producing a target-specific library that comprises substantially all sequences of a pre-determined range of lengths that are comprised within a target polynucleotide sequence, the method comprising:
hybridizing hemi-random probes to a ssDNA target, wherein said hemi-random probes comprise a fixed region comprising primer-binding sequences with encoded restriction enzyme recognition sites and a 10-nt randomized sequence located at the 5′ end in the case of one probe and at the 3′-end in the case of the other;
ligating hybridized probes that hybridize to adjacent target sequences;
amplifying the product of said ligating step;
inserting the product of said amplification into a vector or between DNA sequences allowing expression of the inserted sequences.

21. The method according to claim 2, wherein said vector is an expression vector.

22. The method according to claim 15, wherein said vector is an expression vector.

23. The method according to claim 16, wherein said vector is an expression vector.

24. The method according to claim 20, wherein said vector is an expression vector.


SEQUENCE LISTING (157 sequences)

SEQ ID NO: 1 - RNA, 20 nt, Artificial Sequence
  synthetic primer
gagaauaacaacaacaacaa

SEQ ID NO: 2 - RNA, 19 nt, Artificial Sequence
  synthetic primer
ugguacauuaccugguaac

SEQ ID NO: 3 - DNA, 20 bp, Artificial Sequence
  synthetic primer
ttgttgttgttgttattctc

SEQ ID NO: 4 - DNA, 19 bp, Artificial Sequence
  synthetic primer
tggtacattacctggtaac

SEQ ID NO: 5 - DNA, 36 bp, Artificial Sequence
  synthetic primer
nnnnnnnnnnggatccctgctgacgactagactgtg

SEQ ID NO: 6 - DNA, 36 bp, Artificial Sequence
  synthetic primer
cagtctagcaagtatgcgtcctcgagnnnnnnnnnn

SEQ ID NO: 7 - DNA, 26 bp, Artificial Sequence
  synthetic primer
cacagtctagtcgtcagcagggatcc

SEQ ID NO: 8 - DNA, 26 bp, Artificial Sequence
  synthetic primer
ctcgaggacgcatacttgctagactg

SEQ ID NO: 9 - DNA, 20 bp, Artificial Sequence
  synthetic primer
cacagtctagtcgtcagcag

SEQ ID NO: 10 - DNA, 20 bp, Artificial Sequence
  synthetic primer
cagtctagcaagtatgcgtc

SEQ ID NO: 11 - DNA, 23 bp, Artificial Sequence
  synthetic primer
cttgtggaaagaagcttaaaaag

SEQ ID NO: 12 - DNA, 27 bp, Artificial Sequence
  synthetic primer
agttctgtatgagacagatctaaaaag

SEQ ID NO: 13 - RNA, 10 nt, Artificial Sequence
  synthetic primer
cuuccuguca

SEQ ID NO: 14 - RNA, 10 nt, Artificial Sequence
  synthetic primer
cuuccuguca

SEQ ID NO: 15 - DNA, 21 bp, Artificial Sequence
  synthetic primer
ttctccctcctggctagtccc

SEQ ID NO: 16 - DNA, 21 bp, Artificial Sequence
  synthetic primer
gggagctatttccaggatgtt

SEQ ID NO: 17 - DNA, 21 bp, Artificial Sequence
  synthetic primer
cctttcactcactggcccaag

SEQ ID NO: 18 - DNA, 20 bp, Artificial Sequence
  synthetic primer
catctccctccagaaaagac

SEQ ID NO: 19 - DNA, 22 bp, Artificial Sequence
  synthetic primer
gttccacgtcgcggatcatgct

SEQ ID NO: 20 - DNA, 22 bp, Artificial Sequence
  synthetic primer
gttctggaagccccccatcttt

SEQ ID NO: 21 - DNA, 22 bp, Artificial Sequence
  synthetic primer
agtggggcttcaagtcatctgt

SEQ ID NO: 22 - DNA, 22 bp, Artificial Sequence
  synthetic primer
ttgactactctccctccggtaa

SEQ ID NO: 23 - DNA, 22 bp, Artificial Sequence
  synthetic primer
gctcctccacttggtggtttgc

SEQ ID NO: 24 - DNA, 22 bp, Artificial Sequence
  synthetic primer
gcgctggctcagccactccagc

SEQ ID NO: 25 - DNA, 21 bp, Artificial Sequence
  synthetic primer
aacgccctcctggccaacggc

SEQ ID NO: 26 - DNA, 22 bp, Artificial Sequence
  synthetic primer
acaaccaactagtggtgccagc

SEQ ID NO: 27 - DNA, 22 bp, Artificial Sequence
  synthetic primer
acccatcggctggcaccactag

SEQ ID NO: 28 - DNA, 21 bp, Artificial Sequence
  synthetic primer
cagccgatgggttgtaccttg

SEQ ID NO: 29 - DNA, 22 bp, Artificial Sequence
  synthetic primer
cgatgggttgtaccttgtctac

SEQ ID NO: 30 - DNA, 22 bp, Artificial Sequence
  synthetic primer
aggttctcttcaagggacaagg

SEQ ID NO: 31 - DNA, 22 bp, Artificial Sequence
  synthetic primer
agatagcaaatcggctgacggt

SEQ ID NO: 32 - DNA, 21 bp, Artificial Sequence
  synthetic primer
ccgtcagccgatttgctatct

SEQ ID NO: 33 - DNA, 21 bp, Artificial Sequence
  synthetic primer
cctgagggggctgagctcaaa

SEQ ID NO: 34 - DNA, 22 bp, Artificial Sequence
  synthetic primer
gcagattgacctcagcgctgag

SEQ ID NO: 35 - DNA, 22 bp, Artificial Sequence
  synthetic primer
taagtacttgggcagattgacc

SEQ ID NO: 36 - DNA, 21 bp, Artificial Sequence
  synthetic primer
tagacctgcccggactccgca

SEQ ID NO: 37 - DNA, 22 bp, Artificial Sequence
  synthetic primer
aggggtcagagtaaaggggtca

SEQ ID NO: 38 - DNA, 22 bp, Artificial Sequence
  synthetic primer
tttattgtctactcctcagagc

SEQ ID NO: 39 - DNA, 21 bp, Artificial Sequence
  synthetic primer
tctgtgtccttctaacttaga

SEQ ID NO: 40 - DNA, 22 bp, Artificial Sequence
  synthetic primer
agaaaggggattatggctcaga

SEQ ID NO: 41 - DNA, 21 bp, Artificial Sequence
  synthetic primer
attatggctcagagtccaacc

SEQ ID NO: 42 - DNA, 20 bp, Artificial Sequence
  synthetic primer
gtttgggccagtgagtgaaa

SEQ ID NO: 43 - DNA, 20 bp, Artificial Sequence
  synthetic primer
cctcgagcacgttggacggt

SEQ ID NO: 44 - DNA, 20 bp, Artificial Sequence
  synthetic primer
tcgaattttgagaagatgta

SEQ ID NO: 45 - DNA, 20 bp, Artificial Sequence
  synthetic primer
cggttatgatatgcgcgagg

SEQ ID NO: 46 - DNA, 20 bp, Artificial Sequence
  synthetic primer
ccacataagcacagaaagca

SEQ ID NO: 47 - DNA, 20 bp, Artificial Sequence
  synthetic primer
aagcccgcagccaaccagac

SEQ ID NO: 48 - DNA, 19 bp, Artificial Sequence
  synthetic primer
caaattgttaggatcccac

SEQ ID NO: 49 - DNA, 20 bp, Artificial Sequence
  synthetic primer
tttcgacaccatgagcccag

SEQ ID NO: 50 - DNA, 20 bp, Artificial Sequence
  synthetic primer
ccaaccaagcaggttccgtc

SEQ ID NO: 51 - DNA, 20 bp, Artificial Sequence
  synthetic primer
gatgtcctggccgacggtgt

SEQ ID NO: 52 - DNA, 20 bp, Artificial Sequence
  synthetic primer
cgcgtctgagactcttgcct

SEQ ID NO: 53 - DNA, 20 bp, Artificial Sequence
  synthetic primer
tgccgaatacccttgaactg

SEQ ID NO: 54 - DNA, 16 bp, Artificial Sequence
  synthetic primer
gggtcctttacgttac

SEQ ID NO: 55 - DNA, 20 bp, Artificial Sequence
  synthetic primer
ggtaagagtgggggctgggt

SEQ ID NO: 56 - DNA, 20 bp, Artificial Sequence
  synthetic primer
acgccacatctcccgccaca

SEQ ID NO: 57 - DNA, 18 bp, Artificial Sequence
  synthetic primer
attgaagtacttgtcgag

SEQ ID NO: 58 - DNA, 20 bp, Artificial Sequence
  synthetic primer
gttaacatgtgatgcgggct

SEQ ID NO: 59 - DNA, 20 bp, Artificial Sequence
  synthetic primer
gcgccgtacttgagggcgca

SEQ ID NO: 60 - DNA, 20 bp, Artificial Sequence
  synthetic primer
gcaatgactctaaagtacac

SEQ ID NO: 61 - DNA, 20 bp, Artificial Sequence
  synthetic primer
cttgctaatgactcgaattg

SEQ ID NO: 62 - DNA, 27 bp, Artificial Sequence
  synthetic primer
aaaaagggcgagggcgagggccttttt

SEQ ID NO: 63 - DNA, 33 bp, Artificial Sequence
  synthetic primer
aaaaagtgaaggtgaccaagggcggctcttttt

SEQ ID NO: 64 - DNA, 33 bp, Artificial Sequence
  synthetic primer
aaaaaggcatcctgtccccccagttcccttttt

SEQ ID NO: 65 - DNA, 41 bp, Artificial Sequence
  synthetic primer
aaaaaggcagggaggagtcctgggtcacggtcactcttttt

SEQ ID NO: 66 - DNA, 37 bp, Artificial Sequence
  synthetic primer
aaaaagagacgccgccgtcctcgaagttcatcttttt

SEQ ID NO: 67 - DNA, 37 bp, Artificial Sequence
  synthetic primer
aaaaagaacttcgaggacggcggcgtggtgccttttt

SEQ ID NO: 68 - DNA, 27 bp, Artificial Sequence
  synthetic primer
aaaaagaccacgccgccgtctcttttt

SEQ ID NO: 69 - DNA, 33 bp, Artificial Sequence
  synthetic primer
aaaaagagccgtcctgcagggaggagtcttttt

SEQ ID NO: 70 - DNA, 44 bp, Artificial Sequence
  synthetic primer
aaaaagactcctccctgcaggacggctgcttcatctaccttttt

SEQ ID NO: 71 - DNA, 30 bp, Artificial Sequence
  synthetic primer
aaaaagacacgccgtcgcgggggtcttttt

SEQ ID NO: 72 - DNA, 34 bp, Artificial Sequence
  synthetic primer
aaaaagaggtagtggccgccgtccttcacttttt

SEQ ID NO: 73 - DNA, 27 bp, Artificial Sequence
  synthetic primer
aaaaagacggcggccactactcttttt

SEQ ID NO: 74 - DNA, 40 bp, Artificial Sequence
  synthetic primer
aaaaagacaccatcgtggaggagcgcaccatggtcttttt

SEQ ID NO: 75 - DNA, 42 bp, Artificial Sequence
  synthetic primer
aaaaagggcggccctcggtgcgctcctccacgatggcttttt

SEQ ID NO: 76 - DNA, 22 bp, Artificial Sequence
  synthetic primer
tgagatagcaaatcggctgacg

SEQ ID NO: 77 - DNA, 21 bp, Artificial Sequence
  synthetic primer
ctgacggtgtgggtgaggagt

SEQ ID NO: 78 - DNA, 21 bp, Artificial Sequence
  synthetic primer
ctgacggtgtgggtgaggagc

SEQ ID NO: 79 - DNA, 21 bp, Artificial Sequence
  synthetic primer
ctgacggtgtgggtgaggagc

SEQ ID NO: 80 - DNA, 21 bp, Artificial Sequence
  synthetic primer
ctgacggtgtgggtgaggagc

SEQ ID NO: 81 - DNA, 21 bp, Artificial Sequence
  synthetic primer
aggggtcagagtaaaggggtc

SEQ ID NO: 82 - DNA, 21 bp, Artificial Sequence
  synthetic primer
ttggctgcttgcntttctggg

SEQ ID NO: 83 - DNA, 21 bp, Artificial Sequence
  synthetic primer
ctgacggtgtgggtgaggagc

SEQ ID NO: 84 - DNA, 21 bp, Artificial Sequence
  synthetic primer
gggtgaggagcacgtagtcgg

SEQ ID NO: 85 - DNA, 20 bp, Artificial Sequence
  synthetic primer
ctgacggtgtgggtgaggag

SEQ ID NO: 86 - DNA, 21 bp, Artificial Sequence
  synthetic primer
cgttggccgggagggcgttgg

SEQ ID NO: 87 - DNA, 21 bp, Artificial Sequence
  synthetic primer
cgttggccaggacggcgttgg

SEQ ID NO: 88 - DNA, 21 bp, Artificial Sequence
  synthetic primer
ctgacggtgtgggtgaggagc

SEQ ID NO: 89 - DNA, 21 bp, Artificial Sequence
  synthetic primer
gcaggagggcgttggcgcgct

SEQ ID NO: 90 - DNA, 21 bp, Artificial Sequence
  synthetic primer
ctgacggtgtgggtgaggagc

SEQ ID NO: 91 - DNA, 22 bp, Artificial Sequence
  synthetic primer
ccgttggccaggagggcgttgg

SEQ ID NO: 92 - DNA, 22 bp, Artificial Sequence
  synthetic primer
tgagtgtgagggtctgggccat

SEQ ID NO: 93 - DNA, 21 bp, Artificial Sequence
  synthetic primer
ctgacggtgtgggtgaggagc

SEQ ID NO: 94 - DNA, 20 bp, Artificial Sequence
  synthetic primer
gacggtgtgggtgaggagca

SEQ ID NO: 95 - DNA, 21 bp, Artificial Sequence
  synthetic primer
ctgacggtgtgggtgaggagc

SEQ ID NO: 96 - DNA, 21 bp, Artificial Sequence
  synthetic primer
ctgacggagtgggtgaggagc

SEQ ID NO: 97 - DNA, 22 bp, Artificial Sequence
  synthetic primer
tagaaggacacagactgggggc

SEQ ID NO: 98 - DNA, 21 bp, Artificial Sequence
  synthetic primer
ctgacggtgtgggtgaggagc

SEQ ID NO: 99 - DNA, 22 bp, Artificial Sequence
  synthetic primer
tcttgtagtcggggatgtcggc

SEQ ID NO: 100 - DNA, 19 bp, Artificial Sequence
  synthetic primer
gtagtcggggatgtcggcg

SEQ ID NO: 101 - DNA, 22 bp, Artificial Sequence
  synthetic primer
ctcgttgtgggaggtgatgtcc

SEQ ID NO: 102 - DNA, 21 bp, Artificial Sequence
  synthetic primer
cttgtagtcggggatgtcggc

SEQ ID NO: 103 - DNA, 22 bp, Artificial Sequence
  synthetic primer
cggtaccgtcgactgcagaatt

SEQ ID NO: 104 - DNA, 22 bp, Artificial Sequence
  synthetic primer
tcttgtagtcggggatgtcggc

SEQ ID NO: 105 - DNA, 21 bp, Artificial Sequence
  synthetic primer
acaggatgtcccaggcgaagg

SEQ ID NO: 106 - DNA, 22 bp, Artificial Sequence
  synthetic primer
gtcagganntnncacngcgaag

SEQ ID NO: 107 - DNA, 21 bp, Artificial Sequence
  synthetic primer
ttgtagtcggggaaggtcggc

SEQ ID NO: 108 - DNA, 22 bp, Artificial Sequence
  synthetic primer
attgtngtcggggangtcnnng

SEQ ID NO: 109 - DNA, 21 bp, Artificial Sequence
  synthetic primer
atccggtggatcccgggcccg

SEQ ID NO: 110 - DNA, 21 bp, Artificial Sequence
  synthetic primer
atggtagtggggtatgtgggg

SEQ ID NO: 111 - DNA, 22 bp, Artificial Sequence
  synthetic primer
ctcgttgtgggaggtgatgtcc

SEQ ID NO: 112 - DNA, 21 bp, Artificial Sequence
  synthetic primer
gagccgtactggaactggggg

SEQ ID NO: 113 - DNA, 21 bp, Artificial Sequence
  synthetic primer
cttgntgacgttcttgtngga

SEQ ID NO: 114 - DNA, 19 bp, Artificial Sequence
  synthetic primer
tgtagtcggggatgtcggc

SEQ ID NO: 115 - DNA, 22 bp, Artificial Sequence
  synthetic primer
ctcgttgtgggaggtgatgtcc

SEQ ID NO: 116 - DNA, 21 bp, Artificial Sequence
  synthetic primer
ttgtagtcggggatgtcggcg

SEQ ID NO: 117 - DNA, 22 bp, Artificial Sequence
  synthetic primer
cacttgaagccctcggggaagg

SEQ ID NO: 118 - DNA, 22 bp, Artificial Sequence
  synthetic primer
ctcnntgtgggaggtgatgtcn

SEQ ID NO: 119 - DNA, 22 bp, Artificial Sequence
  synthetic primer
tcgttgtgggaggtgatgtcca

SEQ ID NO: 120 - DNA, 20 bp, Artificial Sequence
  synthetic primer
acaggatgtcccaggcgaag

SEQ ID NO: 121 - DNA, 21 bp, Artificial Sequence
  synthetic primer
tcgttgtgggaggtgatgtcc

SEQ ID NO: 122 - DNA, 22 bp, Artificial Sequence
  synthetic primer
ctcgttgtgggaggtgatgtcc

SEQ ID NO: 123 - DNA, 21 bp, Artificial Sequence
  synthetic primer
atctgagcaggaacaggtggt

SEQ ID NO: 124 - DNA, 11 bp, Artificial Sequence
  synthetic primer
gtcggagggga

SEQ ID NO: 125 - DNA, 22 bp, Artificial Sequence
  synthetic primer
ctcgttgtgggaggtgatgtcc

SEQ ID NO: 126 - DNA, 43 bp, Artificial Sequence
  synthetic primer
aagcttaaaaagnnnnnnnnnnnnnnnnnnnntttttagatct

SEQ ID NO: 127 - DNA, 43 bp, Artificial Sequence
  synthetic primer
ttcgaatttttcnnnnnnnnnnnnnnnnnnnnaaaaatctaga

SEQ ID NO: 128 - DNA, 53 bp, Artificial Sequence
  synthetic primer
aagcttaaaaacgcgtcttcacaacaacaacaacgaagacactttttagatct

SEQ ID NO: 129 - DNA, 53 bp, Artificial Sequence
  synthetic primer
ttcgaatttttgcgcagaagtgttgttgttgttgcttctgtgaaaaatctaga

SEQ ID NO: 130 - DNA, 32 bp, Artificial Sequence
  synthetic primer
aaaaaggttggactctgagccataatcttttt

SEQ ID NO: 131 - DNA, 33 bp, Artificial Sequence
  synthetic primer
aaaaagctctgaggagtagacaataaacttttt

SEQ ID NO: 132 - DNA, 33 bp, Artificial Sequence
  synthetic primer
aaaaagtaagtacttgggcagattgaccttttt

SEQ ID NO: 133 - DNA, 33 bp, Artificial Sequence
  synthetic primer
aaaaagttccacgtcgcggatcatgctcttttt

SEQ ID NO: 134 - DNA, 32 bp, Artificial Sequence
  synthetic primer
aaaaaaagaggctgagacataggcaccttttt

SEQ ID NO: 135 - DNA, 33 bp, Artificial Sequence
  synthetic primer
aaaaagccagggtttgagctcagcccccttttt

SEQ ID NO: 136 - DNA, 42 bp, Artificial Sequence
  synthetic primer
aaaaaccttcacagagcaatgactctacagtagacccttttt

SEQ ID NO: 137 - DNA, 30 bp, Artificial Sequence
  synthetic primer
aaaaagtgcctcttctgccagttccttttt

SEQ ID NO: 138 - DNA, 31 bp, Artificial Sequence
  synthetic primer
aaaaacttggtggtttgctacgacgcttttt

SEQ ID NO: 139 - DNA, 33 bp, Artificial Sequence
  synthetic primer
aaaaagtccacttggtggtttgctacgattttt

SEQ ID NO: 140 - DNA, 32 bp, Artificial Sequence
  synthetic primer
aaaaatcttgacggcagagaggaggtcttttt

SEQ ID NO: 141 - DNA, 33 bp, Artificial Sequence
  synthetic primer
aaaaatctccagctggaagactcctcccttttt

SEQ ID NO: 142 - DNA, 32 bp, Artificial Sequence
  synthetic primer
aaaaagttctccctcctggctagtcccttttt

SEQ ID NO: 143 - DNA, 33 bp, Artificial Sequence
  synthetic primer
aaaaagacccatcggctggcaccactagttttt

SEQ ID NO: 144 - DNA, 32 bp, Artificial Sequence
  synthetic primer
aaaaagatgtggcgccttgggccagtcttttt

SEQ ID NO: 145 - DNA, 34 bp, Artificial Sequence
  synthetic primer
aaaaattaatacgactcactatagggcacttttt

SEQ ID NO: 146 - DNA, 32 bp, Artificial Sequence
  synthetic primer
aaaaaggctcctccacttggtggtttgttttt

SEQ ID NO: 147 - DNA, 44 bp, Artificial Sequence
  synthetic primer
aaaaacggaagttcacgccgatgaacttcaccttgtagattttt

SEQ ID NO: 148 - DNA, 31 bp, Artificial Sequence
  synthetic primer
aaaaaggtgatgtccagcttggaggtttttt

SEQ ID NO: 149 - DNA, 35 bp, Artificial Sequence
  synthetic primer
aaaaacgccgtcggaggggaagttcacgccttttt

SEQ ID NO: 150 - DNA, 35 bp, Artificial Sequence
  synthetic primer
aaaaaggccgccgtccttcagcttcaggggttttt

SEQ ID NO: 151 - DNA, 35 bp, Artificial Sequence
  synthetic primer
aaaaaccctcggggaaggacagcttcttgtttttt

SEQ ID NO: 152 - DNA, 35 bp, Artificial Sequence
  synthetic primer
aaaaaccacttgaatccctcggggaaggacttttt

SEQ ID NO: 153 - DNA, 34 bp, Artificial Sequence
  synthetic primer
aaaaaggtgttgtggccctcgtaggggggttttt

SEQ ID NO: 154 - DNA, 32 bp, Artificial Sequence
  synthetic primer
aaaaacctcgaagttcatcacgcgctcttttt

SEQ ID NO: 155 - DNA, 25 bp, Artificial Sequence
  synthetic primer
aaaaacacgttcttggaggattttt

SEQ ID NO: 156 - DNA, 38 bp, Artificial Sequence
  synthetic primer
aaaaacccttgatgacgttcttggaggagcgcattttt

SEQ ID NO: 157 - DNA, 32 bp, Artificial Sequence
  synthetic primer
aaaaattcacgtacaccttggagccggttttt